(19) United States
(12) Patent Application Publication
(10) Pub. No.: US 2011/0118125 A1
(43) Pub. Date: May 19, 2011

(54) NEONATAL SALIVARY GENOMICS

(75) Inventors: Jill Maron, Sharon, MA (US); Diana Bianchi, Charlestown, MA (US); Kirby Johnson, North Attleboro, MA (US); Donna Slonim, North Andover, MA (US)

(73) Assignees: TUFTS MEDICAL CENTER, INC.; TUFTS UNIVERSITY

(21) Appl. No.: 12/990,855

(22) PCT Filed: May 1, 2009
(86) PCT No.: PCT/US09/42626

§ 371 Date: Feb. 2, 2011

Related U.S. Application Data
(60) Provisional application No. 61/050,213, filed on May 3, 2008.

(51) Int. Cl. C40B 20/04; C12Q 1/68

(52) U.S. Cl. 506/4; 435/6

(57) ABSTRACT

The present invention provides systems for assessing neonatal development and/or conditions by analyzing neonatal saliva RNA. Methods of identifying genes involved in neonatal development and/or conditions affecting neonates, are provided. Methods of determining a diagnosis of a neonate comprising detection of one or more differentially expressed genes are also provided.

### RELATED APPLICATION INFORMATION

The present application claims priority to and benefit of U.S. provisional application Ser. No. 61/050,213, filed on May 3, 2008, the contents of which are herein incorporated by reference in their entirety.

### BACKGROUND

Complications associated with feeding affect the majority of neonates in neonatal intensive care units (NICU). These complications can include gastroesophageal reflux (GERD), feeding intolerance, uncoordinated and immature feeding patterns, and/or inflammatory and necrotic processes of the bowel such as necrotizing enterocolitis (NEC). Such complications often lead to prolonged hospitalizations, medication administration, parental anxiety, and, particularly with NEC, significant neonatal morbidity and mortality.

Development of diagnostics and therapies for such complications has been hindered by the fragility of premature neonates, which excludes them from studies involving invasive procedures. Their limited blood volumes make it impractical or impossible to draw blood from them frequently.

### SUMMARY

The present invention encompasses the recognition that noninvasive yet informative means to monitor the health and disease status and/or development of premature neonates are desirable. The present invention further encompasses the finding that genomic analysis of saliva from neonates may provide the desired noninvasive and informative means. Saliva contains DNA and RNA that can provide useful information. Provided are methods in which saliva is repeatedly obtained from neonates without any danger to the neonates.

In one aspect, the invention provides methods for detecting or identifying genes involved in a condition or disease affecting neonates. In some embodiments, such methods comprise steps of providing a test sample of saliva RNA obtained from a neonate suffering from or diagnosed with a condition; subjecting the test sample of saliva RNA to an analysis, wherein the analysis comprises hybridizing the RNA to one or more oligonucleotide probes, such that one or more genes that are differentially regulated in the sample as compared to a control sample is/are identified, wherein the control sample comprises saliva RNA obtained from a neonate that is not suffering from or diagnosed with the condition; and determining that the one or more differentially regulated genes are involved in the condition or disease. In some embodiments, such methods comprise steps of providing a test sample of saliva RNA obtained from a neonate suffering from or diagnosed with a condition; identifying one or more genes that are differentially regulated in the sample as compared to the control sample, wherein the control sample comprises saliva RNA obtained from a neonate that is not suffering from or diagnosed with the condition; and determining that the one or more differentially regulated genes are involved in the condition or disease. In some embodiments, the condition is necrotizing enterocolitis.

In another aspect, the invention provides methods for detecting or identifying genes involved in neonatal development. In some embodiments, such methods comprise steps of providing a test sample of saliva RNA obtained from a neonate; subjecting the test sample of saliva RNA to an analysis, wherein the analysis comprises hybridizing the RNA to one or more oligonucleotide probes, such that one or more genes that are differentially regulated in the sample as compared to a control sample is/are detected or identified, wherein the control sample comprises saliva RNA obtained from a neonate at a developmental stage different than the neonate from which the test sample of saliva RNA sample was obtained; and determining that the one or more differentially regulated genes are involved in neonatal development. In some embodiments, such methods comprise steps of providing a test sample of saliva RNA obtained from a neonate; identifying one or more genes that are differentially regulated in the sample as compared to a control sample, wherein the control sample comprises saliva RNA obtained from a neonate at a developmental stage different than the neonate from which the test sample of saliva RNA sample was obtained; and determining that the one or more differentially regulated genes are involved in neonatal development. In some embodiments, the developmental stage relates to the neonate's feeding capability. The feeding capability of the neonate can be, for example, the neonate's readiness to feed, feeding tolerance, or both.

In some embodiments, gene expression analyses are used to detect or identify differentially regulated genes. In some such embodiments, the test sample of saliva RNA comprises a plurality of nucleic acid segments labeled with a detectable agent and the step of identifying comprises: providing a gene-expression array comprising a plurality of genetic probes, wherein each genetic probe is immobilized to a discrete spot on a substrate surface to form an array; contacting the array with the test sample under conditions wherein the nucleic acid segments in the sample specifically hybridize to the genetic probes on the array; determining the binding of individual nucleic acid segments of the test sample to individual genetic probes immobilized on the array to obtain a binding pattern; and establishing, based on the binding pattern obtained, a gene expression pattern.

In yet another aspect, the invention provides methods for determining a diagnosis of a neonate. In some embodiments, such methods comprise steps of: providing a sample of saliva RNA obtained from the neonate; subjecting the test sample of saliva RNA to an analysis, wherein the analysis comprises hybridizing the RNA to one or more oligonucleotide probes, such that expression of at least one gene identified using other methods provided in the invention is identified; and determining, based on the detected expression of the at least one gene, a diagnosis of the neonate. In some embodiments, such methods comprise steps of: providing a sample of saliva RNA obtained from the neonate; detecting expression of at least one gene identified using other methods provided in the invention; and determining, based on the detected expression of the at least one gene, a diagnosis of the neonate. In some embodiments, the step of determining the diagnosis comprises determining neonatal developmental progress. In some such embodiments, determining neonatal developmental progress comprises making a determination with respect to a feeding capability of the neonate; in some of these embodiments, the feeding capability is selected from the group consisting of readiness to feed, feeding tolerance, or both. In some embodiments, the step of determining the diagnosis comprises identifying a disease or condition affecting the neonate. In some embodiments, the disease or condition is necrotizing enterocolitis.

In some embodiments, the at least one gene is upregulated. In some embodiments, the at least one gene is down-regulated.

In certain embodiments, inventive methods further comprise detecting at least one gene associated with a disease such as necrotizing enterocolitis. In some such embodiments, the at least one gene is selected from the group consisting of nuclear factor kappa B (NFκB), I kappa B-alpha (IKB-α), toll-like receptor 4 (TLR4), platelet activating factor (PAF), platelet activating factor acetylhydrolase (PAF-AH), interleukin 8 (IL-8), epidermal growth factor (EGF), interleukin 10 (IL-10), endothelial 1 (ET-1), and combinations thereof.

### BRIEF DESCRIPTION OF THE DRAWINGS

FIG. 1 presents a schematic overview of biological markers of interest, their interactions, and their roles in NEC.

FIG. 2 depicts a schematic showing three-dimensional imaging of fetal protein networks. Spheres in red are down-stream proteins of fetal genes detected in the maternal circulation, while yellow spheres are interacting proteins. This schematic overview depicts the intricate network of protein-protein interactions occurring in the developing fetus.

FIG. 3 depicts a representative plot from a BioAnalyzer analysis of amplified total RNA from neonatal saliva sample. Such plots are typically used to evaluate quantity and quality of nucleic acids such as RNA. Time in seconds is plotted on the x-axis and fluorescence is plotted on the y-axis. The area under the curve represents concentration of total RNA extracted from saliva sample. In the BioAnalyzer result depicted, the concentration of amplified total RNA was about 849 ng/μL.

FIG. 4 outlines time points for salivary collection for experiments described in Examples 2-4.

### DEFINITIONS

Throughout the specification, several terms are employed that are defined in the following paragraphs.

As used herein, the terms “about” and “approximately,” in reference to a number, is used herein to include numbers that fall within a range of 20%, 10%, 5%, or 1% in either direction (greater than or less than) the number unless otherwise stated or otherwise evident from the context (except where such number would exceed 100% of a possible value).

As used herein, the term “biomarker” refers to its meaning as understood in the art. The term can refer to an indicator that provides information about, among other things, a process, condition, developmental stage, or outcome of interest, e.g., a neonate's developmental readiness for feeding. In general, the value of such an indicator is correlated with a process, condition, developmental stage, or outcome of interest. The term “biomarker” can also refer to a molecule that is the subject of an assay or measurement the result of which provides information about a process, condition, developmental stage, or outcome of interest. For example, an elevated expression level of a particular gene can be an indicator that a subject has a particular condition. The expression level of the gene, an elevated expression level of the gene, and the gene expression product itself, can all be referred to as “biomarkers.”

As used herein, the term “complementary” refers to nucleic acid sequences that base-pair according to the standard Watson-Crick complementary rules, or that are capable of hybridizing to a particular nucleic acid segment under relatively stringent conditions. Nucleic acid polymers are optionally complementary across only portions of their entire sequences.

As used herein, the term “differentially expressed” in reference to genes refers to the state of having a different expression pattern or level depending on the type of cell, tissue, and/or sample, from which the gene expression products are derived. “Differentially expressed” genes may be upregulated or downregulated in the cell, tissue, and/or samples as compared to controls. For example, a gene that is upregulated in samples obtained from a subject suffering from necrotizing enterocolitis as compared to a subject who is not can be said to be “differentially expressed.” As another example, a gene that is downregulated in samples from a subject that has undergone a developmental transition (such as the ability to swallow) as compared to a subject who has not can also be said to be “differentially expressed.”

As used herein, the term “enteral feeding” refers to delivery of liquid feeding to the gastrointestinal tract via a tube.

As used herein, the phrase “feeding capability” refers collectively to an individual's readiness to feed and feeding tolerance.

As used herein, the phrase “feeding intolerance” refers the inability of an individual (e.g., a neonate) to achieve and/or maintain full enteric feeds. Likewise “feeding tolerance” as used herein refers to the ability of an individual (e.g., a neonate) to achieve and/or maintain full enteric feeds

As used herein, terms “fluorophore”, “fluorescent moiety”, “fluorescent label”, “fluorescent dye” and “fluorescent labeling moiety” are used herein interchangeably. They refer to a molecule that, in solution and upon excitation with light of appropriate wavelength, emits light back. Numerous fluorescent dyes of a wide variety of structures and characteristics are suitable for use in the practice of this invention. Similarly, methods and materials are known for fluorescently labeling nucleic acids (see, for example, Haugland (1994)). In choosing a fluorophore, it is preferred that the fluorescent molecule absorbs light and emits fluorescence with high efficiency (i.e., high molar absorption coefficient and fluorescence quantum yield, respectively) and is photostable (i.e., it does not undergo significant degradation upon light excitation within the time necessary to perform the analysis).

As used herein, the term “gene” refers to a discrete nucleic acid sequence responsible for a discrete cellular product and/or performing one or more intracellular or extracellular functions. In some embodiments, the term “gene” refers to a nucleic acid that includes a portion encoding a protein and optionally encompasses regulatory sequences, such as promoters, enhancers, terminators, and the like, which are involved in the regulation of expression of the protein encoded by the gene of interest. Such gene and regulatory sequences may be derived from the same natural source, or may be heterologous to one another. In some embodiments, a gene does not encode proteins but rather provide templates for transcription of functional RNA molecules such as tRNAs, rRNAs, etc. Alternatively or additionally, in some embodiments, a gene may define a genomic location for a particular event/function, such as the binding of proteins and/or nucleic acids.

As used herein, the term “gene expression” refers to the conversion of the information, contained in a gene, into a gene product. A gene product can be the direct transcriptional product of a gene (e.g., mRNA, tRNA, rRNA, antisense RNA, ribozyme structural RNA or any other type of RNA), or the product of subsequent downstream processing events (e.g., splicing, RNA processing, translation). In some embodiments, a gene product is a protein produced by translation of an mRNA. In some embodiments, gene products are RNAs that are modified by processes such as capping, polyadenylation, methylation, and editing, proteins post-translationally modified, and proteins modified by, for example, methylation, acetylation, phosphorylation, ubiquitination, ADP-ribosylation, myristilation, and glycosylation.

As used herein, the term “gene expression array” refers to an array comprising a plurality of genetic probes immobilized on a substrate surface that can be used for quantitation of mRNA expression levels. In the context of the present invention, the term “array-based gene expression analysis” is used to refer to methods of gene expression analysis that use gene-expression arrays. The term “genetic probe”, as used herein, refers to a nucleic acid molecule of known sequence, which has its origin in a defined region of the genome and can be a short DNA sequence (or oligonucleotide), a PCR product, or mRNA isolate. Genetic probes are gene-specific DNA sequences to which nucleic acids from a test sample of saliva RNA are hybridized. Genetic probes specifically bind (or specifically hybridize) to nucleic acid of complementary or substantially complementary sequence through one or more types of chemical bonds, usually through hydrogen bond formation.

As used herein, the term “gestational age” refers to age of an embryo, fetus, or neonate as calculated from the first day of the mother's last menstrual period. In humans, the gestational age may count the period of time from about two weeks before fertilization takes place.

As used herein, the term “isolated” when applied to RNA means a molecule of RNA or a portion thereof, which (1) by virtue of its origin or manipulation, is separated from at least some of the components with which it was previously associated; or (2) was produced or synthesized by the hand of man.

As used herein, the terms “labeled”, “labeled with a detectable agent” and “labeled with a detectable moiety” are used interchangeably. They are used to specify that a nucleic acid molecule or individual nucleic acid segments from a sample can be visualized, for example, following binding (i.e., hybridization) to genetic probes. In hybridization methods, samples of nucleic acid segments may be detectably labeled before the hybridization reaction or a detectable label may be selected that binds to the hybridization product. Preferably, the detectable agent or moiety is selected such that it generates a signal which can be measured and whose intensity is related to the amount of hybridized nucleic acids. In array-based methods, the detectable agent or moiety is also preferably selected such that it generates a localized signal, thereby allowing spatial resolution of the signal from each spot on the array. Methods for labeling nucleic acid molecules are well known in the art (see below for a more detailed description of such methods). Labeled nucleic acid fragments can be prepared by incorporation of or conjugation to a label, that is directly or indirectly detectable by spectroscopic, photochemical, biochemical, immunochemical, electrical, optical, or chemical means. Suitable detectable agents include, but are not limited to: various ligands, radionuclides, fluorescent dyes, chemiluminescent agents, microparticles, enzymes, colorimetric labels, magnetic labels, and haptens. Detectable moieties can also be biological molecules such as molecular beacons and aptamer beacons.

As used herein, the term “messenger RNA” or “mRNA” refers a form of RNA that serves as a template for protein biosynthesis. In many embodiments, the amount of a particular mRNA (i.e., having a particular sequence, and originating from a particular same gene) reflects the extent to which the gene encoding the mRNA has been “expressed.”

As used herein, the terms “microarray,” “array” and “biochip” are used interchangeably and refer to an arrangement, on a substrate surface, of multiple nucleic acid molecules of known sequences. Each nucleic acid molecule is immobilized to a “discrete spot” (i.e., a defined location or assigned position) on the substrate surface. The term “microarray” more specifically refers to an array that is miniaturized so as to require microscopic examination for visual evaluation. Arrays used in the methods of the invention are preferably microarrays.

As used herein, the abbreviation “NEC” refers necrotizing enterocolitis, a gastrointestinal condition that primarily affects premature neonates and typically involves inflammation, edema, and often perforation and necrosis of the bowel.

As used herein, the terms “neonate,” and “newborn” are used interchangeably and refer to subjects who have recently been born. In some embodiments, the neonate is a human within the first three months of being born. In some embodiments, the neonate is a human within the first two months of being born. In some embodiments, the neonate is a human within the first month of being born. In some embodiments of the invention, the neonate is prematurely born; in some such embodiments, the premature neonate is a human neonate born between 23 and 37 weeks' gestational age.

As used herein, the terms “nucleic acid” and “nucleic acid molecule” are used herein interchangeably. They refer to a deoxyribonucleotide or ribonucleotide polymer in either single- or double-stranded form, and unless otherwise stated, encompass known analogs of natural nucleotides that can function in a similar manner as naturally occurring nucleotides. The terms encompass nucleic acid-like structures with synthetic backbones, as well as amplification products.

As used herein, the term “oligonucleotide” refers to usually short strings of DNA or RNA to be used as hybridizing probes or nucleic acid molecule array elements. These short stretches of sequence are often chemically synthesized. The size of the oligonucleotide depends on the function or use of the oligonucleotides. When used in microarrays for hybridization, oligonucleotides can comprise natural nucleic acid molecules or synthesized nucleic acid molecules and comprise between 5 and 150 nucleotides, preferably between about 15 and about 100 nucleotides, more preferably between 15 and 30 nucleotides and most preferably, between 18 and 25 nucleotides complementary to mRNA.

As used herein, the term “oral feeding” refers to the delivery of feeding to the mouth without the aid of tubes.

As used herein, the terms “premature neonate” and “preterm neonate” are used interchangeably and refer to neonates who are born before the full term of a typical pregnancy. In some embodiments, the premature neonate is a human born at or before 37 weeks' gestation.

As used herein, the term “RNA transcript” refers to the product resulting from transcription of a DNA sequence. When the RNA transcript is the original, unmodified product of a RNA polymerase catalyzed transcription, it is referred to as the primary transcript. An RNA transcript that has been processed (e.g., spliced, etc.) will differ in sequence from the primary transcript; a fully processed transcript is referred to as a “mature” RNA. The term “transcription” refers to the process of copying a DNA sequence of a gene into an RNA product, generally conducted by a DNA-directed RNA polymerase using the DNA as a template. A processed RNA transcript that is translated into protein is often called a messenger RNA (mRNA).

As used herein, the phrase “readiness to feed” refers to a subject's ability to transition from enteral feeding to oral feeding. “Readiness to feed” may be indicative of developmental progress and/or improvement with respect to a medical condition.

As used herein, the term “saliva” refers to a biological fluid produced in and secreted from salivary glands and found in the mouths of humans and other animals. Saliva is comprised of water, digestive enzymes, proteins, hormones, electrolytes, mucus, antibacterial compounds, and nucleic acids DNA and RNA, and is a component of the digestion system. In some embodiments, a saliva sample is obtained by suction from the oropharynx.

As used herein, the term “statistically significant number” refers to a number of samples (analyzed or to be analyzed) that is large enough to provide reliable data.

As used herein, the terms “subject” and “individual” are used herein interchangeably. They refer to a human or another animal (e.g., mouse, rat, rabbit, dog, cat, cattle, swine, sheep, horse, or primate) that can be afflicted with or is susceptible to a disease, disorder, condition, or complication (e.g., necrotizing enterocolitis) but may or may not have the disease or disorder. In many embodiments, the subject is a human being. In many embodiments, the subject is a neonate. In some embodiments, the subject is a premature neonate.

As used herein, the term “susceptible” means having an increased risk for and/or a propensity for something, i.e., a condition such as necrotizing enterocolitis. The term takes into account that an individual “susceptible” for a condition may never be diagnosed with the condition.

### DETAILED DESCRIPTION OF CERTAIN EMBODIMENTS

As mentioned above, the present invention provides technologies for detecting and/or identifying genes that are involved in neonatal development and/or in conditions affecting neonates. The present invention also provides technologies for diagnosing a neonate.

The present inventors have recognized that analyzing neonatal salival RNA may provide valuable information about neonatal development and/or disease. Although some success has been reported in obtaining and analyzing salival RNA from adults, to the knowledge of the present inventors, no attempts have heretofore been made to obtain and analyze salival RNA from neonates. This lack of attempt by others may reflect, among other things, an expectation of failure due to certain difficulties in obtaining and analyzing RNA from neonates. For example, whereas sufficient quantities of saliva for RNA extraction may easily be obtained from adults, much smaller quantities can be obtained from neonates, thus limiting the amount of starting material from which RNA can be obtained. Limited amounts of starting material present challenges for certain analyses, especially those involving large quantities of RNA such as genome-wide gene expression analyses. Such challenges may be exacerbated in premature neonates and/or neonates suffering from a disease or condition, who are often even smaller in size and are often supported by feeding tubes and/or other life support paraphernalia.

The present inventors have overcome these challenges and successfully demonstrated extraction, amplification, and analyses of neonatal salival RNA. In some embodiments, analyses comprise performing genome-wide (“global”) or other large scale gene expression analyses. To the knowledge of the present inventors, such large scale gene expression analyses have heretofore not been performed on any salival RNA samples, as reports on adult salival RNA were limited to analyses of a small subset of genes. Larger scale gene expression analyses on salival RNA, such as those disclosed herein, may provide insight into many physiological and developmental systems and into relationships between gene products. The present inventors have also recognized that profiling gene expression (for example, at a global level) of RNA from developing neonates at various points in time provides further insights.

Such insights, as obtained from methods disclosed herein, are especially valuable for understanding developmental processes relevant to neonates, including those neonates with a disease or condition.

I. Neonatal RNA from Saliva

In some embodiments, methods of the invention involve providing neonatal RNA from saliva samples. Saliva samples can be obtained from neonates by, for example, gentle suctioning of the oropharynx. Typically one can obtain between about 100 μL to about 200 μL saliva by gentle suctioning.

As collecting saliva in this manner is non-invasive, saliva can be collected repeatedly from the same neonate without harm to the neonate. In some embodiments, saliva is collected serially from the same neonate, and in some such embodiments, saliva is collected at various timepoints in a neonate's development.

In some embodiments, saliva is obtained from premature neonates. In some embodiments, saliva is collected from premature neonates that are underdeveloped and/or underweight. Such neonates often have problems relating to feeding, breathing, and/or staying warm. For example, saliva may be collected from human premature neonates that were born at ≦37 weeks' gestation. In some embodiments, saliva is collected from human premature neonates born at ≦32 weeks' gestation. In some embodiments, saliva is collected from human premature neonates born at ≦24 weeks' gestation.

### Isolation of Neonatal Saliva RNA

Neonatal RNA for use in the methods of the present invention is typically isolated from a sample of saliva obtained from a neonate. Such isolation may be carried out by any suitable method of RNA isolation or extraction.

In certain embodiments, neonatal RNA is obtained by treating a sample of saliva such that neonatal RNA present in the sample of saliva is extracted. In some embodiments, neonatal salival RNA is extracted from a sample of saliva containing cells and/or cellular material.

Neonatal RNA may also be obtained by isolating cells from the sample of saliva, optionally cultivating these isolated cells, and extracting RNA from the cells. In such cases, neonatal saliva RNA consists essentially of neonatal RNA from the cultured cells.

In some embodiments, before isolation or extraction of neonatal RNA, the sample of saliva material is stored for a certain period of time under suitable storage conditions. In some embodiments, suitable storage conditions comprise temperatures ranging between about 10° C. to about −220° C., inclusive. In some embodiments, samples are stored at about 4° C., at about −10° C., at about −20° C., at about −70° C., or at about −80° C. In some embodiments, samples are stored for less than about 28 days. In some embodiments, samples are stored for more than about twenty-four hours. In some embodiments, before freezing, an RNase inhibitor, which prevents degradation of neonatal RNA by RNases (i.e., ribonucleases), is added to the sample. In some embodiments, the RNase inhibitor is added within two hours of obtaining the sample of salival material. In some embodiments, the RNAse inhibitor is added within one hour of obtaining the sample of salival material. In some embodiments, the RNAse inhibitor is added within thirty minutes of obtaining the sample of salival material. In some embodiments, the RNAse inhibitor is added within ten minutes of obtaining the sample of salival material. In some embodiments, the RNAse inhibitor is added within five minutes of obtaining the sample of salival material. In some embodiments, the RNAse inhibitor is added within two minutes of obtaining the sample of salival material. In some embodiments, the RNase inhibitor is added immediately after obtaining the sample of remaining salival material. In some embodiments, before RNA extraction, the frozen sample is thawed at 37° C. and mixed with a vortex.

In some embodiments, the sample is frozen (e.g., flash-frozen in liquid nitrogen and dry ice), stored, and thawed; then RNAse inhibitor is added after thawing. In some such embodiments, the RNase inhibitor is added within two hours of thawing. In some embodiments, the RNAse inhibitor is added within one hour of thawing. In some embodiments, the RNAse inhibitor is added within thirty minutes of thawing. In some embodiments, the RNAse inhibitor is added within ten minutes of thawing. In some embodiments, the RNAse inhibitor is added within five minutes of thawing. In some embodiments, the RNAse inhibitor is added within two minutes of thawing.

The most commonly used RNase inhibitor is a natural protein derived from human placenta that specifically (and reversibly) binds RNases (Blackburn et al. (1977), the entire contents of which are herein incorporated by reference),). RNase inhibitors are commercially available, for example, from Ambion (Austin, Tex.; as SUPERase•In™), Promega, Inc. (Madison, Wis.; as rRNasin® Ribonuclease Inhibitor) and Applied Biosystems (Framingham, Mass.). In general, precautions for preventing RNases contaminations in RNA samples, which are well known in the art and include the use of gloves, of certified RNase-free reagents and ware, of specifically treated water and of low temperatures, as well as routine decontamination and the like, are used in the practice of the methods of the invention.

Isolating neonatal RNA may include treating the remaining salival material such that neonatal RNA present in the remaining salival material is extracted and made available for analysis. Any suitable isolation method that results in extracted saliva neonatal RNA may be used in the practice of the invention. In order to obtain the most accurate assessment of the neonate, it is desirable to minimize artifacts from manipulation processes. Therefore, the number of extraction and modification steps is in some embodiments kept as low as possible.

Methods of RNA extraction are well known in the art (see, for example, Sambrook et al. (1989). Most methods of RNA isolation from bodily fluids or tissues are based on the disruption of the tissue in the presence of protein denaturants to quickly and effectively inactivate RNases. Generally, RNA isolation reagents comprise, among other components, guanidinium thiocyanate and/or beta-mercaptoethanol, which are known to act as RNase inhibitors (Chirgwin et al. (1979)). Isolated total RNA is then further purified from the protein contaminants and concentrated by selective ethanol precipitations, phenol/chloroform extractions followed by isopropanol precipitation (see, for example, Chomczynski and Sacchi (1987)) or cesium chloride, lithium chloride or cesium trifluoroacetate gradient centrifugations (see, for example, Glisin et al (1974) and Stern and Newton (1986)).

In certain methods of the invention, for example those wherein saliva neonatal RNA is subjected to a gene-expression analysis, it may be desirable to isolate mRNA from total RNA in order to allow the detection of even low level messages (Alberts et al. (1994)).

Purification of mRNA from total RNA typically relies on the poly(A) tail present on most mature eukaryotic mRNA species. Several variations of isolation methods have been developed based on the same principle. In a first approach, a solution of total RNA is passed through a column containing oligo(dT) or d(U) attached to a solid cellulose matrix in the presence of high concentrations of salts to allow the annealing of the poly(A) tail to the oligo(dT) or d(U). The column is then washed with a lower salt buffer to remove and release the poly(A) mRNAs. In a second approach, a biotinylated oligo(dT) primer is added to the solution of total RNA and used to hybridize to the 3′ poly(A) region of the mRNAs. The hybridization products are captured and washed at high stringency using streptavidin coupled to paramagnetic particles and a magnetic separation stand. The mRNA is eluted from the solid phase by the simple addition of ribonuclease-free deionized water. Other approaches do not require the prior isolation of total RNA. For example, uniform, superparamagnetic, polystyrene beads with oligo(dT) sequences covalently bound to the surface may be used to isolate mRNA directly by specific base pairing between the poly(A) residues of mRNA and the oligo(dT) sequences on the beads. Furthermore, the oligo(dT) sequence on the beads may also be used as a primer for the reverse transcriptase to subsequently synthesize the first strand of cDNA. Alternatively, new methods or improvements of existing methods for total RNA or mRNA isolation, preparation and purification may be devised by one skilled in the art and used in the practice of the methods of the invention.

Numerous different and versatile kits can be used to extract RNA (i.e., total RNA or mRNA) from bodily fluids and are commercially available from, for example, Ambion, Inc. (Austin, Tex.), Amersham Biosciences (Piscataway, N.J.), BD Biosciences Clontech (Palo Alto, Calif.), BioRad Laboratories (Hercules, Calif.), Dynal Biotech Inc.(Lake Success, N.Y.), Epicentre Technologies (Madison, Wis.), Gentra Systems, Inc. (Minneapolis, Minn.), GIBCO BRL (Gaithersburg, Md.), Invitrogen Life Technologies (Carlsbad, Calif.), MicroProbe Corp. (Bothell, Wash.), Organon Teknika (Durham, N.C.), Promega, Inc. (Madison, Wis.), and Qiagen Inc. (Valencia, Calif.). For example, theRNAprotect Saliva Kit (Qiagen) may be used to extract salival RNA. User Guides that describe in great detail the protocol to be followed are usually included in all these kits. Sensitivity, processing time and cost may be different from one kit to another. One of ordinary skill in the art can easily select the kit(s) most appropriate for a particular situation.

### Amplification of Extracted Neonatal Saliva RNA

In certain embodiments, the saliva neonatal RNA is amplified before being analyzed. In some embodiments, before analysis, the saliva neonatal RNA is converted, by reverse-transcriptase, into complementary DNA (cDNA), which, optionally, may, in turn, be converted into complementary RNA (cRNA) by transcription.

Amplification methods are well known in the art (see, for example, Kimmel and Berger (1987), Sambrook et al (1989), Ausubel (Ed.) (2002), and U.S. Pat. Nos. 4,683,195; 4,683,202 and 4,800,159). Standard nucleic acid amplification methods include: polymerase chain reaction (or PCR, see, for example, Innis (Ed.) (1990) and Innis (Ed.) (1995)) and ligase chain reaction (or LCR, see, for example, Landegren et al. (1988); and Barringer (1990)).

Methods for transcribing RNA into cDNA are also well known in the art. Reverse transcription reactions may be carried out using non-specific primers, such as an anchored oligo-dT primer, or random sequence primers, or using a target-specific primer complementary to the RNA for each genetic probe being monitored, or using thermostable DNA polymerases (such as avian myeloblastosis virus reverse transcriptase or Moloney murine leukemia virus reverse transcriptase). Other methods include transcription-based amplification system (TAS) (see, for example, Kwoh et al. (1989)), isothermal transcription-based systems such as Self-Sustained Sequence Replication (3SR) (see, for example, Guatelli et al. (1990)), and Q-beta replicase amplification (see, for example, Smith et al. (1997); and Burg et al., (1996)).

The cDNA products resulting from these reverse transcriptase methods may serve as templates for multiple rounds of transcription by the appropriate RNA polymerase (for example, by nucleic acid sequence based amplification or NASBA, see, for example, Kievits et al. (1991), and Greijer et al. (2001)). Transcription of the cDNA template rapidly amplifies the signal from the original target mRNA.

In some embodiments, nucleic acid amplification methods designed to amplify from limited biological material (e.g., from a single cell) and/or from the entire transcriptome are used. (Amplification of the entire transcriptome may be particularly desirable for global gene expression analyses.) For example, NuGEN Technologies's (www.nugeninc.com) RNA amplification systems are suitable for use in the practice of the invention and are described in U.S. Pat. Nos. 6,692,918; 6,251,639; 6,946,251 (the contents of which are herein incorporated by reference in their entirety). NuGEN amplification systems include, but are not limited to, WT-Ovation™ RNA Amplification System, WT-Ovation™ Pico RNA Amplification System, WT-Ovation™ FFPE System V2, and Ovation® RNA Amplification System V2. With NuGEN's Ribo-SPIA™ technology, amplification of target RNA molecules is initiated at both the 3′ end and randomly througout the transcriptome using a first strand DNA/RNA chimeric primer mix and reverse transcriptase (RT). Microgram quantities of cDNA can be prepared from as little as 500 pg to 50 ng total RNA.

These methods as well as others (either known or newly devised by one skilled in the art) may be used in the practice of the invention.

Amplification can also be used to quantify the amount of extracted neonatal RNA (see, for example, U.S. Pat. No. 6,294,338). Alternatively or additionally, amplification using appropriate oligonucleotide primers can be used to label cell-free neonatal RNA prior to analysis (see below). Suitable oligonucleotide amplification primers can easily be selected and designed by one skilled in the art.

### Labeling of Neonatal Saliva RNA

In certain embodiments, neonatal saliva RNA (for example, after amplification, or after conversion to cDNA or to cRNA) is labeled with a detectable agent or moiety before being analyzed. The role of a detectable agent is to facilitate detection of neonatal RNA or to allow visualization of hybridized nucleic acid fragments (e.g., nucleic acid fragments bound to genetic probes). In some embodiments, the detectable agent is selected such that it generates a signal which can be measured and whose intensity is related to the amount of labeled nucleic acids present in the sample being analyzed. In array-based analysis methods, the detectable agent is also in some embodiments selected such that it generates a localized signal, thereby allowing spatial resolution of the signal from each spot on the array.

The association between the nucleic acid molecule and detectable agent can be covalent or non-covalent. Labeled nucleic acid fragments can be prepared by incorporation of or conjugation to a detectable moiety. Labels can be attached directly to the nucleic acid fragment or indirectly through a linker. Linkers or spacer arms of various lengths are known in the art and are commercially available, and can be selected to reduce steric hindrance, or to confer other useful or desired properties to the resulting labeled molecules (see, for example, Mansfield et al. (1995)).

Methods for labeling nucleic acid molecules are well-known in the art. For a review of labeling protocols, label detection techniques and recent developments in the field (see, for example, Kricka (2002), van Gijlswijk et al. (2001), and Joos et al. (1994)). Standard nucleic acid labeling methods include: incorporation of radioactive agents, direct attachment of fluorescent dyes (see, for example, Smith et al. (1985)) or of enzymes (see, for example, Connoly and Rider (1985)); chemical modifications of nucleic acid fragments making them detectable immunochemically or by other affinity reactions (see, for example, Broker et al. (1978), Bayer et al., (1980), Langer et al. (1981), Richardson et al. (1983), Brigati et al. (1983), Tchen et al. (1984), Landegent et al. (1984), and Hopman et al. (1987)); and enzyme-mediated labeling methods, such as random priming, nick translation, PCR and tailing with terminal transferase (for a review on enzymatic labeling, see, for example, Temsamani and Agrawal (1996)). More recently developed nucleic acid labeling systems include, but are not limited to: ULS (Universal Linkage System; see, for example, Wiegant et al. (1999)), photoreactive azido derivatives (see, for example, Neves et al. (2000)), and alkylating agents (see, for example, Sebestyen et al. (1998)).

Any of a wide variety of detectable agents can be used in the practice of the present invention. Suitable detectable agents include, but are not limited to: various ligands, radionuclides (such as, for example, ³²P, ³⁵S, ³H, ¹⁴C, ¹²⁵I, ¹³¹I and the like); fluorescent dyes (for specific exemplary fluorescent dyes, see below); chemiluminescent agents (such as, for example, acridinium esters, stabilized dioxetanes and the like); microparticles (such as, for example, quantum dots, nanocrystals, phosphors and the like); enzymes (such as, for example, those used in an ELISA, i.e., horseradish peroxidase, beta-galactosidase, luciferase, alkaline phosphatase); colorimetric labels (such as, for example, dyes, colloidal gold and the like); magnetic labels (such as, for example, Dynabeads™); and biotin, dioxigenin or other haptens and proteins for which antisera or monoclonal antibodies are available.

In certain embodiments, neonatal saliva RNA (after amplification, or conversion to cDNA or to cRNA) is fluorescently labeled. Numerous known fluorescent labeling moieties of a wide variety of chemical structures and physical characteristics are suitable for use in the practice of this invention. Suitable fluorescent dyes include, but are not limited to: Cy-3™, Cy-5™, Texas red, FITC, phycoerythrin, rhodamine, fluorescein, fluorescein isothiocyanine, carbocyanine, merocyanine, styryl dye, oxonol dye, BODIPY dye (i.e., boron dipyrromethene difluoride fluorophore, see, for example, Chen et al. (2000), Chen et al. (2000), U.S. Pat. Nos. 4,774,339; 5,187,288; 5,227,487; 5,248,782; 5,614,386; 5,994,063; and 6,060,324), and equivalents, analogues, derivatives or combinations of these molecules. Similarly, methods and materials are known for linking or incorporating fluorescent dyes to biomolecules such as nucleic acids (see, for example, Haugland (1994)). Fluorescent labeling dyes as well as labeling kits are commercially available from, for example, Amersham Biosciences, Inc. (Piscataway, N.J.), Molecular Probes, Inc. (Eugene, Oreg.), and New England Biolabs, Inc. (Beverly, Mass.).

Favorable properties of fluorescent labeling agents to be used in the practice of the invention include high molar absorption coefficient, high fluorescence quantum yield, and photostability. Some labeling fluorophores exhibit absorption and emission wavelengths in the visible (i.e., between 400 and 750 nm) rather than in the ultraviolet range of the spectrum (i.e., lower than 400 nm).

In other embodiments, neonatal saliva RNA (for example, after amplification or conversion to cDNA or cRNA) is made detectable through one of the many variations of the biotin-avidin system, which are well known in the art. Biotin RNA labeling kits are commercially available, for example, from Roche Applied Science (Indianapolis, Ind.) Perkin Elmer (Boston, Mass.), and NuGEN (San Carlos, Calif.).

Detectable moieties can also be biological molecules such as molecular beacons and aptamer beacons. Molecular beacons are nucleic acid molecules carrying a fluorophore and a non-fluorescent quencher on their 5′ and 3′ ends. In the absence of a complementary nucleic acid strand, the molecular beacon adopts a stem-loop (or hairpin) conformation, in which the fluorophore and quencher are in close proximity to each other, causing the fluorescence of the fluorophore to be efficiently quenched by FRET (i.e., fluorescence resonance energy transfer). Binding of a complementary sequence to the molecular beacon results in the opening of the stem-loop structure, which increases the physical distance between the fluorophore and quencher thus reducing the FRET efficiency and allowing emission of a fluorescence signal. The use of molecular beacons as detectable moieties is well-known in the art (see, for example, Sokol et al. (1998); and U.S. Pat. Nos. 6,277,581 and 6,235,504). Aptamer beacons are similar to molecular beacons except that they can adopt two or more conformations (see, for example, Kaboev et al. (2000), Yamamoto et al. (2000), Hamaguchi et al. (2001), and Poddar and Le (2001)).

A “tail” of normal or modified nucleotides may also be added to nucleic acid fragments for detectability purposes. A second hybridization with nucleic acid complementary to the tail and containing a detectable label (such as, for example, a fluorophore, an enzyme or bases that have been radioactively labeled) allows visualization of the nucleic acid fragments bound to the array (see, for example, system commercially available from Enzo Biochem Inc., New York, N.Y.).

The selection of a particular nucleic acid labeling technique will depend on the situation and will be governed by several factors, such as the ease and cost of the labeling method, the quality of sample labeling desired, the effects of the detectable moiety on the hybridization reaction (e.g., on the rate and/or efficiency of the hybridization process), the nature of the detection system to be used, the nature and intensity of the signal generated by the detectable label, and the like.

II. Analysis of Neonatal RNA from Saliva

According to the present invention, neonatal saliva RNA can be analyzed to obtain information regarding the neonatal RNA. In certain embodiments, analyzing the neonatal saliva RNA comprises determining the quantity, concentration or sequence composition of neonatal RNA.

Neonatal saliva RNA may be analyzed by any of a variety of methods. Methods of analysis of RNA are well-known in the art (see, for example, Sambrook et al. (1989) and Ausubel (Ed.) (2002)).

For example, the quantity and concentration of neonatal RNA extracted from saliva may be evaluated by UV spectroscopy, wherein the absorbance of a diluted RNA sample is measured at 260 and 280 nm (Wilfinger et al. (1997)). Quantitative measurements may also be carried out using certain fluorescent dyes, such as, for example, RiboGreen® (commercially available from Molecular Probes, Eugene, Oreg.), which exhibit a large fluorescence enhancement when bound to nucleic acids. RNA labeled with these fluorescent dyes can be detected using standard fluorometers, fluorescence microplate reader or filter fluorometers. Another method for analyzing quantity and quality of RNA samples is through use of a BioAnalyzer (commercially available from Agilent Technologies, Foster City, Calif.), which separates charged biological molecules (such as nucleic acids) using microfluidic technologies and then a laser to excite intercalating fluorescent dyes.

Neonatal saliva RNA may also be analyzed through sequencing. For example, RNase T1, which cleaves single-stranded RNA specifically at the 3′-side of guanosine residues in a two-step mechanism, may be used to digest denatured RNA. Partial digestion of 3′ or 5′ labeled RNA with this enzyme thus generates a ladder of G residues. The cleavage can be monitored by radioactive (Ikehara et al. (1986)) and photometric (Grunert et al (1993)) detection systems, by zymogram assay (Bravo et al. (1994)), agar diffusion test (Quaas et al. (1989)), lanthan assay (Anfinsen et al. (1954)) or methylene blue test (Greiner-Stoeffele et al. (1996)) or by fluorescence correlation spectroscopy (Korn et al. (2000)).

Other methods for analyzing neonatal saliva RNA include northern blots, wherein the components of the RNA sample being analyzed are resolved by size prior to detection thereby allowing identification of more than one species simultaneously, and slot/dot blots, wherein unresolved mixtures are used.

In certain embodiments, analyzing the neonatal saliva RNA comprises submitting the extracted RNA to a gene-expression analysis. In some embodiments, this includes the simultaneous analysis of multiple genes.

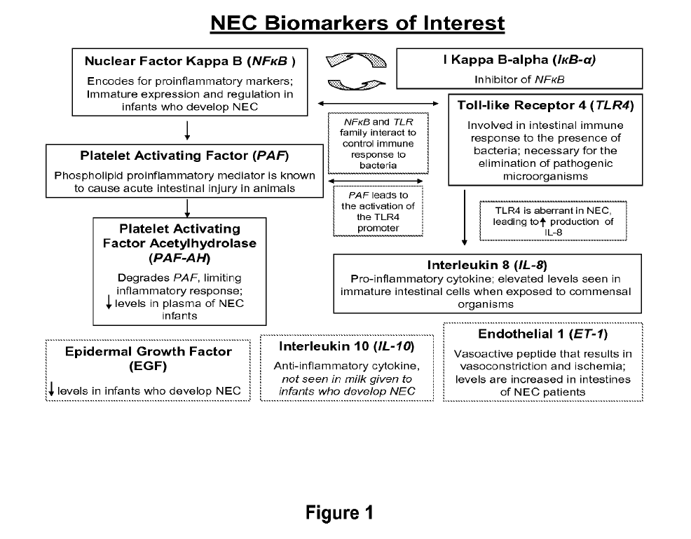

For example, analysis of neonatal saliva RNA may include detecting the presence of and/or quantitating a neonatal RNA transcribed from a gene known to be involved in NEC. Some examples of such genes are presented in FIG. 1 and include, but are not limited to, nuclear factor kappa B (NFκB), I kappa B-alpha (IκB-α), toll-like receptor 4 (TLR4), platelet activating factor (PAF), platelet activating factor acetylhydrolase (PAF-AH), interleukin 8 (IL-8), epidermal growth factor (EGF), interleukin 10 (IL-10), endothelial 1 (ET-1), and combinations thereof.

As another example, analysis of neonatal saliva RNA may include detection of the presence of and/or quantitating RNA transcribed from genes that are involved in feeding and digestion. These include genes encoding digestive enzymes such as luminal enterokinase, lactase, carboyxpeptidase D., etc.

Analysis of neonatal saliva RNA may include detection of the presence of RNA transcribed from mesenchymal developmental genes, neurodevelopmental genes, cytokines, and immunoglobulins. These genes include neurturin, glial cell derived neurotrophic factor, B-cell CLL/Lymphoma 2, etc. As another example, detection of and/or determining expression levels of surfactant genes may be used as a way of monitoring neonatal lung development.

In analyses carried out to detect the presence or absence of RNA transcribed from a specific gene, the detection may be performed by any of a variety of physical, immunological and biochemical methods. Such methods are well-known in the art, and include, for example, protection from enzymatic degradation such as S1 analysis and RNase protection assays, in which hybridization to a labeled nucleic acid probe is followed by enzymatic degradation of single-stranded regions of the probe and analysis of the amount and length of probe protected from degradation.

In some embodiments of the invention, real time RT-PCR methods are employed that allow quantification of RNA transcripts and viewing of the increase in amount of nucleic acid as it is amplified. The TaqMan assay, a quenched fluorescent dye system, may also be used to quantitate targeted mRNA levels (see, for example Livak et al. (1995)).

In some embodiments of the invention involving methods that allow quantification of RNA transcripts (such as real time RT-PCR), expression housekeeping genes are used as normalization controls. Examples of housekeeping genes include GAPDH, 18S rRNA, beta-actin, cyclophilin, tubulin, etc.

Other methods are based on the analysis of cDNA derived from mRNA, which is less sensitive to degradation than RNA and therefore easier to handle. These methods include, but are not limited to, sequencing cDNA inserts of an expressed sequence tag (EST) clone library (see, for example, Adams et al. (1991)) and serial analysis of gene expression (or SAGE), which allows quantitative and simultaneous analysis of a large number of transcripts (see, for example, U.S. Pat. No. 5,866,330; Velculescu et al. (1995); and Zhang et al. (1997)). These two methods survey the whole spectrum of mRNA in a sample rather than focusing on a predetermined set.

Other methods of analysis of cDNA derived from mRNA include reverse transcriptase-mediated PCR(RT-PCR) gene expression assays. These methods are directed at specific target gene products and allow the qualitative (non-quantitative) detection of transcripts of very low abundance (see, for example, Su et al. (1997)). A variation of these methods, called competitive RT-PCR, in which a known amount of exogenous template is added as internal control, has been developed to allow quantitative measurements (see, for example, Becker-Andre and Hahlbrock (1989), Wang (1989), and Gilliland et al. (1990)).

mRNA analysis may also be performed by differential display reverse transcriptase PCR (DDRT-PCR; see, for example, Liang and Pardee (1992)) or RNA arbitrarily primed PCR (RAP-CPR; see, for example, Welsh et al. (1992) and McClelland et al. (1993)). In these methods, RT-PCR fingerprint profiles of transcripts are generated by random priming and differentially expressed genes appear as changes in the fingerprint profiles between two samples. Identification of a differentially expressed gene requires further manipulation (i.e., the appropriate band of the gel must be excised, subcloned, sequenced and matched to a gene in a sequence database).

### III. Array-Based Gene Expression Analysis of Neonatal Saliva RNA

In certain embodiments, the methods of the invention include submitting neonatal saliva RNA to an array-based gene expression analysis.

### Array-Based Gene Expression Analysis

Traditional molecular biology methods, such as most of those described above, typically assess one gene per experiment, which significantly limits the overall throughput and prevents gaining a broad picture of gene function. Technologies based on DNA array or microarray (also called gene expression microarray), which were developed more recently, offer the advantage of allowing the monitoring of thousands of genes simultaneously through identification of sequence (gene/gene mutation) and determination of gene expression level (abundance) of genes (see, for example, Marshall and Hodgson (1998), Ramsay, (1998), Ekins and Chu (1999), and Lockhart and Winzeler (2000)).

In a gene expression experiment, labeled cDNA or cRNA targets derived from the mRNA of an experimental sample are hybridized to nucleic acid probes immobilized to a solid support. By monitoring the amount of label associated with each DNA location, it is possible to infer the abundance of each mRNA species represented.

There are two standard types of DNA microarray technology in terms of the nature of the arrayed DNA sequence. In the first format, probe cDNA sequences (typically 500 to 5,000 bases long) are immobilized to a solid surface and exposed to a plurality of targets either separately or in a mixture. In the second format, oligonucleotides (typically 20-80-mer oligos) or peptide nucleic acid (PNA) probes are synthesized either in situ (i.e., directly on-chip) or by conventional synthesis followed by on-chip attachment, and then exposed to labeled samples of nucleic acids.

The analyzing step in the methods of the invention can be performed using any of a variety of methods, means and variations thereof for carrying out array-based gene expression analysis. Array-based gene expression methods are known in the art and have been described in numerous scientific publications as well as in patents (see, for example, Schena et al. (1995), Schena et al. (1996), and Chen et al. (1998); U.S. Pat. Nos. 5,143,854; 5,445,934; 5,807,522; 5,837,832; 6,040,138; 6,045,996; 6,284,460; and 6,607,885)

In the practice of the present invention, these methods as well as other methods known in the art for carrying out array-based gene expression analysis may be used as described or modified such that they allow neonatal mRNA levels of gene expression to be evaluated.

### Test Sample

In some embodiments, neonatal saliva RNA to be analyzed by an array-based gene expression method is isolated from a sample of saliva as described above. A test sample of neonatal saliva RNA to be used in the methods of the invention may include a plurality of nucleic acid fragments labeled with a detectable agent.

The extracted neonatal RNA may be amplified, reverse-transcribed, labeled, fragmented, purified, concentrated and/or otherwise modified prior to the gene-expression analysis. Techniques for the manipulation of nucleic acids are well-known in the art, see, for example, Sambrook et al., (1989), Innis (Ed.) (1990), Tijssen (1993), Innis (Ed.) (1995), and Ausubel (Ed.) (2002).

In certain embodiments, in order to improve the resolution of the array-based gene expression analysis, the nucleic acid fragments of the test sample are less then 500 bases long, in some embodiments less than about 200 bases long. The use of small fragments significantly increases the reliability of the detection of small differences or the detection of unique sequences.

Methods of RNA fragmentation are known in the art and include: treatment with ribonucleases (e.g., RNase T1, RNase V1 and RNase A), sonication (see, for example, Deininger (1983)), mechanical shearing, and the like (see, for example, Sambrook et al. (1989), Tijssen (1993), Ordahl et al. (1976), Oefner et al. (1996), Thorstenson et al. (1998)). Random enzymatic digestion of the RNA leads to fragments containing as low as 25 to 30 bases.

Fragment size of the nucleic acid segments in the test sample may be evaluated by any of a variety of techniques, such as, for example, electrophoresis (see, for example, Siles and Collier (1997)) or matrix-assisted laser desorption/ionization time-of-flight mass spectrometry (see, for example, Chiu et al. (2000)).

In the practice of the methods of the invention, the test sample of neonatal saliva RNA is labeled before analysis. Suitable methods of nucleic acid labeling with detectable agents have been described in detail above.

Prior to hybridization, the labeled nucleic acid fragments of the test sample may be purified and concentrated before being resuspended in the hybridization buffer. Columns such as Microcon 30 columns may be used to purify and concentrate samples in a single step. Alternatively or additionally, nucleic acids may be purified using a membrane column (such as a Qiagen column) or Sephadex G50 and precipitated in the presence of ethanol.

### Gene-Expression Hybridization Arrays

Any of a variety of arrays may be used in the practice of the present invention. Investigators can either rely on commercially available arrays or generate their own. Methods of making and using arrays are well known in the art (see, for example, Kern and Hampton, (1997), Schummer et al., (1997), Solinas-Toldo et al. (1997), Johnston (1998), Bowtell (1999), Watson and Akil (199), Freeman et al. (2000), Lockhart and Winzeler (2000), Cuzin (2001), Zarrinkar et al., (2001), Gabig and Wegrzyn, (2001), and Cheung et al. (2001); see also, for example, U.S. Pat. Nos. 5,143,854; 5,434,049; 5,556,752; 5,632,957; 5,700,637; 5,744,305; 5,770,456; 5,800,992; 5,807,522; 5,830,645; 5,856,174; 5,959,098; 5,965,452; 6,013,440; 6,022,963; 6,045,996; 6,048,695; 6,054,270; 6,258,606; 6,261,776; 6,277,489; 6,277,628; 6,365,349; 6,387,626; 6,458,584; 6,503,711; 6,516,276; 6,521,465; 6,558,907; 6,562,565; 6,576,424; 6,587,579; 6,589,726; 6,594,432; 6,599,693; 6,600,031; and 6,613,893).

Arrays comprise a plurality of genetic probes immobilized to discrete spots (i.e., defined locations or assigned positions) on a substrate surface. Gene arrays used in accordance with some embodiments of the invention contain probes representing a comprehensive set of genes across the genome. In some such embodiments, the genes represented by the probes do not represent any particular subset of genes, and/or may be a random assortment of genes. In some embodiments of the invention, the gene arrays comprise a particular subset or subsets of genes. The subsets of genes may be represent particular classes of genes of interest. For example, an array comprising probes for developmental genes may be used in order to focus analyses on developmental genes. In such embodiments using arrays having particular subsets, more than one class of genes of interest may be represented on the same array.

Substrate surfaces suitable for use in the present invention can be made of any of a variety of rigid, semi-rigid or flexible materials that allow direct or indirect attachment (i.e., immobilization) of genetic probes to the substrate surface. Suitable materials include, but are not limited to: cellulose (see, for example, U.S. Pat. No. 5,068,269), cellulose acetate (see, for example, U.S. Pat. No. 6,048,457), nitrocellulose, glass (see, for example, U.S. Pat. No. 5,843,767), quartz or other crystalline substrates such as gallium arsenide, silicones (see, for example, U.S. Pat. No. 6,096,817), various plastics and plastic copolymers (see, for example, U.S. Pat. Nos. 4,355,153; 4,652,613; and 6,024,872), various membranes and gels (see, for example, U.S. Pat. No. 5,795,557), and paramagnetic or supramagnetic microparticles (see, for example, U.S. Pat. No. 5,939,261). When fluorescence is to be detected, arrays comprising cyclo-olefin polymers may in some embodiments be used (see, for example, U.S. Pat. No. 6,063,338).

The presence of reactive functional chemical groups (such as, for example, hydroxyl, carboxyl, amino groups and the like) on the material can be exploited to directly or indirectly attach genetic probes to the substrate surface. Methods for immobilizing genetic probes to substrate surfaces to form an array are well-known in the art.

More than one copy of each genetic probe may be spotted on the array (for example, in duplicate or in triplicate). This arrangement may, for example, allow assessment of the reproducibility of the results obtained. Related genetic probes may also be grouped in probe elements on an array. For example, a probe element may include a plurality of related genetic probes of different lengths but comprising substantially the same sequence. Alternatively, a probe element may include a plurality of related genetic probes that are fragments of different lengths resulting from digestion of more than one copy of a cloned piece of DNA. A probe element may also include a plurality of related genetic probes that are identical fragments except for the presence of a single base pair mismatch. An array may contain a plurality of probe elements. Probe elements on an array may be arranged on the substrate surface at different densities.

Array-immobilized genetic probes may be nucleic acids that contain sequences from genes (e.g., from a genomic library), including, for example, sequences that collectively cover a substantially complete genome or a subset of a genome (for example, the array may contain only human genes that are expressed throughout development). Genetic probes may be long cDNA sequences (500 to 5,000 bases long) or shorter sequences (for example, 20-80-mer oligonucleotides). The sequences of the genetic probes are those for which gene expression levels information is desired. Additionally or alternatively, the array may comprise nucleic acid sequences of unknown significance or location. Genetic probes may be used as positive or negative controls (for example, the nucleic acid sequences may be derived from karyotypically normal genomes or from genomes containing one or more chromosomal abnormalities; alternatively or additionally, the array may contain perfect match sequences as well as single base pair mismatch sequences to adjust for non-specific hybridization).

Techniques for the preparation and manipulation of genetic probes are well-known in the art (see, for example, Sambrook et al. (1989), Innis (Ed.) (1990), Tijssen (1993), Innis (Ed.) (1995), and Ausubel (Ed.) (2002)).

Long cDNA sequences may be obtained and manipulated by cloning into various vehicles. They may be screened and re-cloned or amplified from any source of genomic DNA. Genetic probes may be derived from genomic clones including mammalian and human artificial chromosomes (MACs and HACs, respectively, which can contain inserts from ˜5 to 400 kilobases (kb)), satellite artificial chromosomes or satellite DNA-based artificial chromosomes (SATACs), yeast artificial chromosomes (YACs; 0.2-1 Mb in size), bacterial artificial chromosomes (BACs; up to 300 kb); P1 artificial chromosomes (PACs; ˜70-100 kb) and the like.

Genetic probes may also be obtained and manipulated by cloning into other cloning vehicles such as, for example, recombinant viruses, cosmids, or plasmids (see, for example, U.S. Pat. Nos. 5,266,489; 5,288,641 and 5,501,979).

In some embodiments, genetic probes are synthesized in vitro by chemical techniques well-known in the art and then immobilized on arrays. Such methods are especially suitable for obtaining genetic probes comprising short sequences such as oligonucleotides and have been described in scientific articles as well as in patents (see, for example, Narang et al. (1979), Brown et al. (1979), Belousov et al. (1997), Guschin et al. (1997), Blommers et al., (1994) and Frenkel et al. (1995); see also for example, U.S. Pat. No. 4,458,066).

For example, oligonucleotides may be prepared using an automated, solid-phase procedure based on the phosphoramidite approach. In such a method, each nucleotide is individually added to the 5-end of the growing oligonucleotide chain, which is attached at the 3′-end to a solid support. The added nucleotides are in the form of trivalent 3′-phosphoramidites that are protected from polymerization by a dimethoxytrityl (or DMT) group at the 5-position. After base-induced phosphoramidite coupling, mild oxidation to give a pentavalent phosphotriester intermediate and DMT removal provides a new site for oligonucleotide elongation. The oligonucleotides are then cleaved off the solid support, and the phosphodiester and exocyclic amino groups are deprotected with ammonium hydroxide. These syntheses may be performed on commercial oligo synthesizers such as the Perkin Elmer/Applied Biosystems Division DNA synthesizer.

Methods of attachment (or immobilization) of oligonucleotides on substrate supports have been described (see, for example, Maskos and Southern (1992), Matson et al. (1995), Lipshutz et al. (1999), Rogers et al. (1999), Podyminogin et al. (2001), Belosludtsev et al. (2001)).

Oligonucleotide-based arrays have also been prepared by synthesis in situ using a combination of photolithography and oligonucleotide chemistry (see, for example, Pease et al. (1994), Lockhart et al. (1996), Singh-Gasson et al. (1999), Pirrung et al. (2001), McGall et al., (2001), Barone et al. (2001), Butler et al. (2001), Nuwaysir et al. (2002)). The chemistry for light-directed oligonucleotide synthesis using photolabile protected 2′-deoxynucleoside phosphoramites has been developed by Affymetrix Inc.(Santa Clara, Calif.) and is well known in the art (see, for example, U.S. Pat. Nos. 5,424,186 and 6,582,908).

An alternative to custom arraying of genetic probes is to rely on commercially available arrays and micro-arrays. Such arrays have been developed, for example, by Affymetrix Inc. (Santa Clara, Calif.), Illumina, Inc. (San Diego, Calif.), Spectral Genomics, Inc. (Houston, Tex.), and Vysis Corporation (Downers Grove, Ill.).

### Hybridization

In the methods of the invention, the gene expression array may be contacted with the test sample under conditions wherein the nucleic acid fragments in the sample specifically hybridize to the genetic probes immobilized on the array.

The hybridization reaction and washing step(s), if any, may be carried out under any of a variety of experimental conditions. Numerous hybridization and wash protocols have been described and are well-known in the art (see, for example, Sambrook et al. (1989), Tijssen (1993), Innis (Ed.) (1995), and Anderson (Ed.) (1999)). The methods of the invention may be carried out by following known hybridization protocols, by using modified or optimized versions of known hybridization protocols or newly developed hybridization protocols as long as these protocols allow specific hybridization to take place.

The term “specific hybridization” refers to a process in which a nucleic acid molecule preferentially binds, duplexes, or hybridizes to a particular nucleic acid sequence under stringent conditions. In the context of the present invention, this term more specifically refers to a process in which a nucleic acid fragment from a test sample preferentially binds (i.e., hybridizes) to a particular genetic probe immobilized on the array and to a lesser extent, or not at all, to other immobilized genetic probes of the array. Stringent hybridization conditions are sequence dependent. The specificity of hybridization increases with the stringency of the hybridization conditions; reducing the stringency of the hybridization conditions results in a higher degree of mismatch being tolerated.

The hybridization and/or wash conditions may be adjusted by varying different factors such as the hybridization reaction time, the time of the washing step(s), the temperature of the hybridization reaction and/or of the washing process, the components of the hybridization and/or wash buffers, the concentrations of these components as well as the pH and ionic strength of the hybridization and/or wash buffers.

In certain embodiments, the hybridization and/or wash steps are carried out under very stringent conditions. In other embodiments, the hybridization and/or wash steps are carried out under moderate to stringent conditions. In still other embodiments, more than one washing steps are performed. For example, in order to reduce background signal, a medium to low stringency wash is followed by a wash carried out under very stringent conditions.

As is well known in the art, the hybridization process may be enhanced by modifying other reaction conditions. For example, the efficiency of hybridization (i.e., time to equilibrium) may be enhanced by using reaction conditions that include temperature fluctuations (i.e., differences in temperature that are higher than a couple of degrees). An oven or other devices capable of generating variations in temperatures may be used in the practice of the methods of the invention to obtain temperature fluctuation conditions during the hybridization process.

It is also known in the art that hybridization efficiency is significantly improved if the reaction takes place in an environment where the humidity is not saturated. Controlling the humidity during the hybridization process provides another means to increase the hybridization sensitivity. Array-based instruments usually include housings allowing control of the humidity during all the different stages of the experiment (i.e., pre-hybridization, hybridization, wash and detection steps).

Additionally or alternatively, a hybridization environment that includes osmotic fluctuation may be used to increase hybridization efficiency. Such an environment where the hyper-/hypo-tonicity of the hybridization reaction mixture varies may be obtained by creating a solute gradient in the hybridization chamber, for example, by placing a hybridization buffer containing a low salt concentration on one side of the chamber and a hybridization buffer containing a higher salt concentration on the other side of the chamber

### Highly Repetitive Sequences

In the practice of the methods of the invention, the array may be contacted with the test sample under conditions wherein the nucleic acid segments in the sample specifically hybridize to the genetic probes on the array. As mentioned above, the selection of appropriate hybridization conditions will allow specific hybridization to take place. In certain cases, the specificity of hybridization may further be enhanced by inhibiting repetitive sequences.

In certain embodiments, repetitive sequences present in the nucleic acid fragments are removed or their hybridization capacity is disabled. By excluding repetitive sequences from the hybridization reaction or by suppressing their hybridization capacity, one prevents the signal from hybridized nucleic acids to be dominated by the signal originating from these repetitive-type sequences (which are statistically more likely to undergo hybridization). Failure to remove repetitive sequences from the hybridization or to suppress their hybridization capacity results in non-specific hybridization, making it difficult to distinguish the signal from the background noise.

Removing repetitive sequences from a mixture or disabling their hybridization capacity can be accomplished using any of a variety of methods well-known to those skilled in the art. These methods include, but are not limited to, removing repetitive sequences by hybridization to specific nucleic acid sequences immobilized to a solid support (see, for example, Brison et al. (1982)); suppressing the production of repetitive sequences by PCR amplification using adequate PCR primers; or inhibiting the hybridization capacity of highly repeated sequences by self-reassociation (see, for example, Britten et al. (1974)).

In some embodiments, the hybridization capacity of highly repeated sequences is competitively inhibited by including, in the hybridization mixture, unlabeled blocking nucleic acids. The unlabeled blocking nucleic acids, which are mixed to the test sample before the contacting step, act as a competitor and prevent the labeled repetitive sequences from binding to the highly repetitive sequences of the genetic probes, thus decreasing hybridization background. In certain embodiments, for example when cDNA derived from neonatal mRNA is analyzed, the unlabeled blocking nucleic acids are Human Cot-1 DNA. Human Cot-1 DNA is commercially available, for example, from Gibco/BRL Life Technologies (Gaithersburg, Md.).

### Binding Detection and Data Analysis

In some embodiments, inventive methods include determining the binding of individual nucleic acid fragments of the test sample to individual genetic probes immobilized on the array in order to obtain a binding pattern. In array-based gene expression, determination of the binding pattern is carried out by analyzing the labeled array that results from hybridization of labeled nucleic acid segments to immobilized genetic probes.

In certain embodiments, determination of the binding includes: measuring the intensity of the signals produced by the detectable agent at each discrete spot on the array.

Analysis of the labeled array may be carried out using any of a variety of means and methods, whose selection will depend on the nature of the detectable agent and the detection system of the array-based instrument used.

In certain embodiments, the detectable agent comprises a fluorescent dye and the binding is detected by fluorescence. In other embodiments, the sample of neonatal saliva RNA is biotin-labeled and after hybridization to immobilized genetic probes, the hybridization products are stained with a streptavidin-phycoerythrin conjugate and visualized by fluorescence. Analysis of a fluorescently labeled array usually comprises: detection of fluorescence over the whole array, image acquisition, quantitation of fluorescence intensity from the imaged array, and data analysis.

Methods for the detection of fluorescent labels and the creation of fluorescence images are well known in the art and include the use of “array reading” or “scanning” systems, such as charge-coupled devices (i.e., CCDs). Any known device or method, or variation thereof can be used or adapted to practice the methods of the invention (see, for example, Hiraoka et al., (1987), Aikens et al. (1989), Divane et al. (1994), Jalal et al. (1998), and Cheung et al. (1999); see also, for example, U.S. Pat. Nos. 5,539,517; 5,790,727; 5,846,708; 5,880,473; 5,922,617; 5,943,129; 6,049,380; 6,054,279; 6,055,325; 6,066,459; 6,140,044; 6,143,495; 6,191,425; 6,252,664; 6,261,776 and 6,294,331).

Commercially available microarrays scanners are typically laser-based scanning systems that can acquire one (or more) fluorescent image (such as, for example, the instruments commercially available from PerkinElmer Life and Analytical Sciences, Inc. (Boston, Mass.), Virtek Vision, Inc. (Ontario, Canada) and Axon Instruments, Inc. (Union City, Calif.)). Arrays can be scanned using different laser intensities in order to ensure the detection of weak fluorescence signals and the linearity of the signal response at each spot on the array. Fluorochrome-specific optical filters may be used during the acquisition of the fluorescent images. Filter sets are commercially available, for example, from Chroma Technology Corp. (Rockingham, Vt.).

In some embodiments, a computer-assisted imaging system capable of generating and acquiring fluorescence images from arrays such as those described above, is used in the practice of the methods of the invention. One or more fluorescent images of the labeled array after hybridization may be acquired and stored.

In some embodiments, a computer-assisted image analysis system is used to analyze the acquired fluorescent images. Such systems allow for an accurate quantitation of the intensity differences and for an easier interpretation of the results. A software for fluorescence quantitation and fluorescence ratio determination at discrete spots on an array is usually included with the scanner hardware. Softwares and/or hardwares are commercially available and may be obtained from, for example, BioDiscovery (El Segundo, Calif.), Imaging Research (Ontario, Canada), Affymetrix, Inc. (Santa Clara, Calif.), Applied Spectral Imaging Inc. (Carlsbad, Calif.); Chroma Technology Corp. (Brattleboro, Vt.); Leica Microsystems, (Bannockburn, Ill.); and Vysis Inc. (Downers Grove, Ill.). Other softwares are publicly available (e.g., MicroArray Image Analysis, and Combined Expression Data and Sequence Analysis (http://rana.lbl.gov); Chiang et al. (2001); a system written in R and available through the Bioconductor project (http://www.bioconductor.org); a Java-based TM4 software system available from the Institute for Genomic Research (http://www.tigr.org/software); and a Web-based system developed at Lund University (http: base.thep.lu.se)).

Accurate determination of fluorescence intensities often requires normalization and determination of the fluorescence ratio baseline (Brazma and Vilo (2000)). Data reproducibility may be assessed by using arrays on which genetic probes are spotted in duplicate or triplicate. Baseline thresholds may also be determined using global normalization approaches (M. K. Kerr et al. (2000)). Other arrays include a set of maintenance genes which shows consistent levels of expression over a wide variety of tissues and allows the normalization and scaling of array experiments.

In the practice of the methods of the invention, any of a large variety of bioinformatics and statistical methods may be used to analyze data obtained by array-based gene expression analysis. Such methods are well known in the art (for a review of essential elements of data acquisition, data processing, data analysis, data mining and of the quality, relevance and validation of information extracted by different bioinformatics and statistical methods, see, for example, Watson et al. (1998), Duggan et al. (1999), Bassett et al. (1999), Hess et al. (2001), Marcotte and Date (2001), Weinstein et al. (2002), Dewey (2002), Butte (2002), Tamames et al. (2002), Xiang et al. (2003).

### IV. Gene Expression Patterns and Neonatal Health and Disease

### Methods of Detecting or Identifying Genes

In certain aspects, the invention provides methods of detecting or identifying genes of interest in neonatal health and disease. Provided methods include methods for detecting or identifying genes involved in neonatal development. Such methods comprise providing a neonatal saliva RNA sample, identifying differentially expressed genes (as compared to appropriate control samples), and determining that the differentially expressed genes are involved in neonatal development. Also provided are methods for detecting identifying genes involved in a condition or disease affecting neonates. Such methods comprise providing a neonatal saliva RNA sample, identifying differentially expressed genes (as compared to appropriate control samples, such as from neonates not diagnosed with the condition or disease), and determining that the differentially expressed genes are involved in the condition or disease or disease.

### Identifying Differentially Expressed Genes

A variety of methods of detecting gene expression have been described herein. Differentially expressed genes are genes whose expression level differs depending on the cell, tissue, and/or sample from which the gene products are obtained. Genes may be identified as differentially expressed through gene expression array experiments using microarrays. Such methods have been described herein and are also described in Examples 2-5. In such experiments, genes are identified as differentially expressed in comparison with a control. The choice of an appropriate control depends on what kinds of genes one would like to identify.

To detect or identify genes involved in neonatal development, for example, one may compare gene expression data from test samples with data from control samples obtained from neonates who are at a different developmental stage than neonates from whom the test samples were obtained. As will be understood by those of skill in the art, a variety of criteria may be used in determining developmental stage. In some embodiments of the invention, developmental stage is assessed with respect to factors such as body weight. In some embodiments of the invention, developmental stage is assessed with respect to feeding capabilities, e.g., readiness to feed and/or feeding tolerance. In some embodiments of the invention, developmental stage is assessed with respect to gestational age. In some embodiments of the invention, developmental stage is assessed with respect to capability of breathing without assistance, coordination of breathing rhythms, etc. In some embodiments of the invention, developmental stage is assessed with respect to a combination of factors, including combinations of any of the afore-mentioned factors. As another example, to detect or identify genes involved in a condition or disease affecting neonates, one may compare gene expression data from a cohort of neonates suffering from or diagnosed with a condition (e.g., necrotizing enterocolitis) with data from a cohort of neonates who do not suffer from or are diagnosed with that condition.

Methods of determining levels of gene expression have already been described herein. In gene expression array experiments, quantitative readouts of expression levels are typically provided. Typically, after normalization of data, genes having at least a 1.5-fold differences (i.e. a ratio of about 1.5) in expression levels between test and control samples may be considered “differentially expressed.” In some embodiments of the invention, genes considered to be differentially expressed show at least two-fold, at least five-fold, at least ten-fold, at least 15-fold, at least 20-fold, or at least 25-fold different expression levels compared to controls. (It is to be understood that the fold different expression levels can be determined in either direction, i.e., the expression levels for the test sample may be at least 1.5-fold higher or 1.5-fold lower than expression levels for the control sample.)

It will be appreciated, however, that both the fold-difference cutoff for being considered differentially expressed varies depending on several factors which may include, for example, the type of samples used, the quantity and quality of the RNA sample, the power of the statistical analyses, the type of genes of interest, etc. In some embodiments, a lower cutoff ratio (i.e. —fold difference) is used, e.g., ratios of about 1.4, or about 1.37. In some embodiments, a higher cutoff ratio than about 1.5 is used, e.g., about 2.5, about 3.0, about 3.5, about 4.0, about 4.5, about 5.0, etc.

In some embodiments of the invention, a preliminary list of genes is identified as being differentially expressed using a particular statistical method or particular set of experimental data. In some embodiments, the preliminary list is narrowed down. That is, genes are identified within the preliminary list. Determining which genes among the preliminary list may be done in a hypothesis-driven manner. For example, only genes on the preliminary list that are deemed to be physiologically relevant (as determined, by example, by what is known of the gene's function, localization, structure, etc.) may be ultimately identified as differentially expressed genes of interest. In some embodiments, genes are identified within the preliminary list without regard to a particular hypothesis. A subset of genes from the preliminary list may be identified as genes of interest using, for example, a different method of gene expression analysis, a different set of samples. etc. In some embodiments, no further selection or identification of genes is done after obtaining the preliminary list of genes.

It will be understood that inventive methods may identify some genes that are not known, not previously described in the literature, and/or not catalogued in publicly available databases. For example, some gene expression microarrays may contain probes for genes that have not yet been characterized or known in the literature. In cases in which uncharacterized genes are identified as being differentially regulated, the genes may still be described as being “identified” because there is usually an identifier, e.g., a probe with a known sequence on the microarray that can be associated with the gene, a name of an expressed sequence tag, etc.

Determining that Genes are Involved in Development or in a Condition or Disease

In some embodiments, determining that the genes identified as being differentially expressed are involved in the developmental process, condition, or disease of interest comprises deciding that genes meeting a particular cutoff for differential expression are involved. In some embodiments, determining that the genes are involved comprises one or more further steps. These further steps may involve alternative methods to determine gene expression such as those described herein, assessment of the gene's function, etc. Assessment of the gene's function may involve any or a any combination of analyzing literature on the gene, analyzing information on the gene in gene databases (e.g., OMIM, http://www.ncbi.nlm.nih.gov/sites/entrez?db=OMIM; PubMed, https://www.ncbi.nlm.nih.gov/sites/entrez; NetAffx, http://www.affymetrix.com/analysis/index.affx; UniGene, http://www.ncbi.nlm.nih.gov.sites/entrez?db=unigene; Ingenuity®, http://wwww.ingenuity.com etc.), performing additional experiments that may elucidate the gene's function, (e.g., genetic, biochemical, structural, etc.) etc.

### Methods of Diagnosing

In some aspects, the invention provides methods of determining a diagnosis of a neonate. Such methods comprise steps of providing a sample of saliva RNA obtained from the neonate; detecting expression of at least one gene identified as being differentially expressed using other methods of the invention, and determining, based on the detected expression of the at least one gene, a diagnosis of the neonate.

### Genes

Products of genes identified in other inventive methods may be used as markers in diagnostic methods of the invention. Some of these genes have been identified by experiments described in Example 4. In some embodiments of the invention, expression of one or more genes selected from the group consisting of glutamate-cysteine ligase, catalytic subunit, CD3d, cholecytokinin A receptor, fibroblast growth receptor 2, arginase liver and combinations thereof is detected and/or identified. In some embodiments, expression of one or more genes upregulated during neonatal development is detected. In some such embodiments, expression of one or more genes selected from the group consisting of neuropeptide Y receptor Y1 (NPY1R); leptin receptor (LEPR); growth hormone secretagogue receptor (GHSR); prostaglandin E receptor 3 (subtype EP3) (PTGER 3); hypocretin (orexin) receptor 2 (HCRTR2); galanin receptor 3 (GALR3); lactalbumin alpha (LALBA); glucagon (GCG); melanin-concentrating hormone receptor 1 (MCHR1); prostaglandin E receptor 3 (PTGER3); cholecytokinin A receptor (CCKAR); odorant binding protein 2B (OBP2B); transient receptor potential cation channel, subfamily V, member 1 (TRPV1); taste receptor, type 2, member 1 (TAS2R1); surfactant protein B (SFTPB); cystic fibrosis transmembrane conductance regulator (CFTR); fibroblast growth factors (FGF) 1, 2, 7, 10, 18; fibroblast growth receptor 2 (FGFR2); and combinations thereof is detected and/or identified.

In some embodiments, expression of one or more genes downregulated during neonatal development is detected. In some such embodiments, expression of one or more genes selected from the group consisting of carcinoembryonic antigen-related cell adhesion molecule 1 (biliary glycoprotein) (CEACAM1); V-raf murine sarcoma viral oncogene homolog B1 (BRAF); amino-terminal enhancer of split (AES); E1A binding protein p300 (EP300); Fas (TNF receptor superfamily member 6) (FAS); Fas (TNFRSF6)-associated via death domain (FADD); cyclin-dependent kinase inhibitor 2A (melanoma, p16, inhibits CDK4) (CDKN2A); glycogen synthase kinase 3 Beta (GSK3B); protein kinase, cAMP-dependent, regulatory, type 1, alpha (tissue specific extinguisher 1) (PRKAR1A); signal transducer and activator of transcription 5B (STAT5B); aryl hydrocarbon receptor nuclear translocator (ARNT); insulin receptor (INSR); and combinations thereof is detected and/or identified.

In some embodiments, expression of genes from the aforementioned list and/or genes identified using methods of the invention is used together with expression of known genes involved in particular processes to determine a diagnosis.

In some embodiments, expression of genes previously known to be involved in NEC are also detected and used in a determination of the relevant diagnosis. In some embodiments, expression of one or more genes selected from the group consisting of (NKκB), I kappa B-alpha (IκB-α), toll-like receptor 4 (TLR4), platelet activating factor (PAF), platelet activating factor acetylhydrolase (PAF-AH), interleukin 8 (IL-8), epidermal growth factor (EGF), interleukin 10 (IL-10), endothelial 1 (ET-1), and combinations thereof are also detected and/or identified.

### Diagnoses

Determining a diagnosis of a neonate may involve making a determination with respect to the developmental progress of the neonate. Developmental progress may relate to such factors as the neonate's feeding capabilities, such as readiness to feed (readiness to transition from enteral feeding to oral feeding) and/or feeding tolerance (ability to establish and/or maintain full enteral feeding). Developmental progress may be assessed in relation to other factors such as ability to breathe independently and/or with a coordinated rhythm, etc.

Determining a diagnosis of a neonate can involve, among other things, determining that the neonate is susceptible for a condition or disease, that the neonate is developing the condition or disease, that the neonate has the condition or disease, that the neonate has a particular stage of the condition or disease, and/or that the neonate's condition is improving or recovering from a disease.

The condition or disease that may be determined may relate to problems of development, neurodevelopment, breathing, feeding, etc. For example, the disease may relate to problems in the digestive system, which may be underdeveloped in the neonate, and which relate to feeding. Such conditions or disease often affect prematurely born neonates. In some embodiments of the invention, the condition or disease that is determined is selected from the group consisting of necrotizing enterocolitis, respiratory distress syndrome, bronchopulmonary dysplasia, sepsis, and combinations thereof.

(1) In some embodiments, the condition is necrotizing enterocolitis (NEC). In some such embodiments, a determination is made that the neonate has one of the following stages of NEC according to the Bell classification: IA (suspected); IB (suspected with bloody stool); IIA (definite, mildly ill); IIB (definite, moderately ill); IIIA (advanced, severely ill, intact bowel); and IIIB (advanced, severely ill, perforated bowel). It will be recognized that although the Bell classification is typically used to stage NEC, other staging systems may be used in accordance with the invention.

### EXAMPLES

The following examples describe some of the preferred modes of making and practicing the present invention. However, it should be understood that these examples are for illustrative purposes only and are not meant to limit the scope of the invention. Furthermore, unless the description in an Example is presented in the past tense, the text, like the rest of the specification, is not intended to suggest that experiments were actually performed or data were actually obtained.

### Example 1

### Using Genomic Databases to Clinically Correlate Gene Lists with the Developing Fetus

The inventors had previously established a library of information regarding normal fetal gene expression at term. In the present Example, this list of fetal biomarkers detected in the maternal circulation was clinically correlated to a developing fetus. It was recognized that to achieve this goal, known gene functions and tissue expression patterns for each gene had to be identified. Therefore, the present inventors navigated through publicly available genomic databases such as Gene Ontology (GO), UniGene, Pubmed, and NetAffx. The expanded knowledge about the fetal biomarkers as well as the databases will be extremely important in genomic analyses of premature neonates.

### Example 2

### Data Mining of Gene Lists to Determine Relevant Biologic Networks

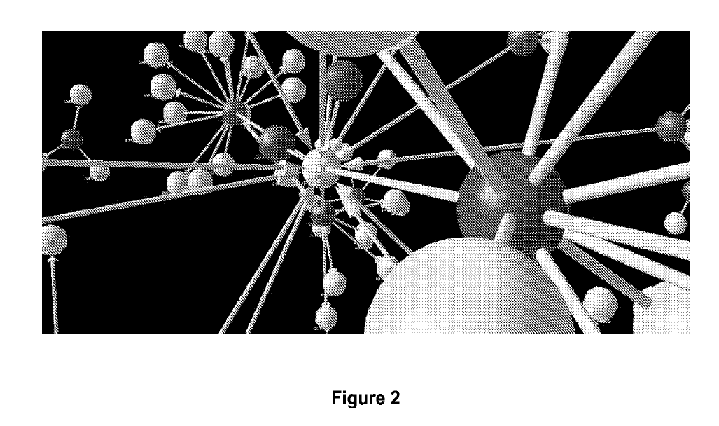

Using a program developed by Dr. Gil Alterovitz known as Massome, proteins encoded by the initial fetal gene transcripts were woven into intricate networks of fetal protein-protein interactions (see FIG. 2). In-depth analysis at this level continues to yield important information. For example, the inventors had initially speculated that the overabundance of immune system genes was an essential defense mechanism for the fetus as it transitioned from its in utero environment. Protein analysis of these immune genes identified specific proteins that appeared to be linked directly to the fetus. These included HIST1H2BK, which is believed to be responsible for the bactericidal activity of amniotic fluid. Other specific proteins such as CREBZF, which suppresses the expression of HSV proteins in cells infected with the virus, appear to pinpoint the exact role of the gene target. Similarly, protein analysis of the genes in an initial list of nervous system transcripts (identified in previous experiments) can now clarify which aspect of the nervous system appears to be overrepresented in the healthy term fetus. Glial cell differentiation and development, oligodendrocyte differentiation and development, and gliogenesis are all over-represented in the present protein network analysis (all p-values <0.01). Such analyses confirm how this additional layer of genetic pinpointing precise gene functions, protein network analysis will also help direct future therapy targets in pregnancies complicated by aberrant fetal development. To the best of the inventor's knowledge, this work represents to first protein network analysis of the fetus. The inventors anticipate performing similar analyses on neonatal salivary genomic data to develop protein networks of the developing ill preterm infant.

### Example 3

### Identification of Appropriate Housekeeping Genes for Normalization of Gene Expression Data

As mentioned previously, genes that appear to be associated with either a protective or harmful effect on neonatal feeding pathology are of particular interest to the present inventors. Expression of such genes will be confirmed using real time RT-PCR. Relative quantification of expression levels using real time RT-PCR requires choosing an appropriate housekeeping gene whose expression levels can be used to normalize data.

In this Example, expression of housekeeping genes including GAPDH, 18S rRNA, beta-actin, and cyclophilin A was analyzed in newborn cord blood. Beta-actin was identified as a suitable housekeeping gene for normalization of data from newborn blood samples. For designing real time RT-PCR assays on neonatal salivary samples, similar analyses will be performed to determine suitable housekeeping gene or genes.

### Examples 4-7

### Gene Expression Analyses on Neonatal Saliva Samples

Whole transcriptome microarrays are used in each of Examples 2-5. Although the analyses in the following Examples are initially focused on neonatal feeding and related complications, data generated from the Examples help build a library of banked neonatal genomic information. Development of this library is a long term goal of the experiments described below. Such a library may provide an invaluable resource for retrospective focused analyses of different neonatal complications and may contribute to our overall understanding of neonatal developmental genomic and network pathways.

### Example 4

### Gene Expression Analyses on Neonatal Saliva Samples and Identification of Genes Involved in Feeding

The experiments described in this Example illustrate that RNA can be successfully extracted and amplified from neonatal saliva samples and used in gene expression profiling experiments. Furthermore, experiments in this Example identified a limited list of genes whose expressions were differentially regulated in neonates who were feeding (at time of sample collection) compared those who were not. Among the list of differentially expressed genes are genes encoding digestive enzymes and neurodevelopmental genes. These results confirm that gene expression profiling of saliva samples can uncover physiologically relevant genes and suggest that biomarkers involved in particular processes, disease states, and/or conditions can be identified using such methods. Specific hypotheses relating to the involvement of particular genes or types of genes in such processes, disease states, and/or conditions may be tested using experimental paradigms similar to those used in this Example.

To date, 36 neonates ranging in gestational ages from about 27 5/7 weeks to 34 1/7 weeks have been enrolled in this study, and 109 salivary samples have been obtained by suctioning from the neonate's oropharynx. Each sample comprised approximately 100-200 μL of saliva. Two infants developed NEC after having been prospectively enrolled before onset of the disease.

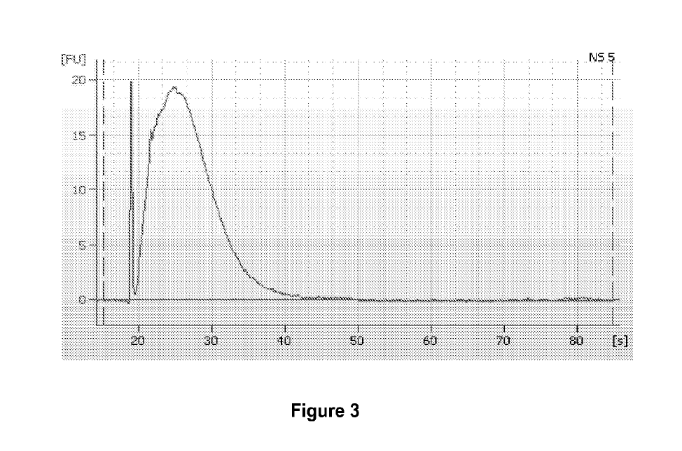

Total RNA was extracted from each sample and stored at −80° C. until further use. As depicted in FIG. 3, neonatal salival RNA was successfully amplified in quantities more than sufficient for further experiments, demonstrating that extracted RNA was of high quality. FIG. 3 shows representative BioAnalyzer result of amplified total RNA from neonatal saliva sample. Following amplification, concentrations of starting RNA material ranged from about 600 ng/μL to about 3,200 ng/μL.

Five infants were selected for microarray analyses. These infants had a relatively benign neonatal course and did not have significant gastrointestinal sequelae. Their pertinent clinical information can be found in Table 1.

TABLE 1
clinical characteristics of subjects selected
for initial microarray analyses

| | | Gestational age at | Birth weight |
|---|---|---|---|
| Subject | Gender | birth (weeks) | (grams) |
| 1 | Male | 29 0/7 | 1389 |
| 2 | Female | 28 3/7 | 942 |
| 3 | Male | 28 3/7 | 1123 |
| 4 | Female | 32 0/7 | 1683 |
| 5 | Female | 32 0/7 | 1379 |

For each infant, five microarrays were run from salivary RNA obtained from the following time points: 1) shortly after birth and prior to enteral feeds, 2) at initiation of enteral feeds, 3) at full enteral nutrition, 4) at start of oral feeding, and 5) at full or majority oral feeding. For each sample, 5 μg of amplified and labeled RNA was hybridized onto an Affymetrix HG U133 Plus 2.0 whole genomic microarray. Hybridization rates for arrays ranged from about 7% to about 32%. Calculations were done in R version 2.8.1, a computer language program within Bioconductor version 2.3 (Gentleman et al. (2004), the entire contents of which are herein incorporated by reference) and lme4 (Bates et al., the entire contents of which are herein incorporated by reference). (For more information about R, see the website whose address is “http:” followed immediately by “//www.r-project.org/”.) Probe sets were summarized and arrays normalized using the rma( ) function in the Bioconductor affy package with default settings (Gautier et al. (2004), the entire contents of which are herein incorporated by reference). For each probe set, the significance of gestational age was determined by fitting two statistical models. The first model fit a random subject effect. The second model fit a linear age effect and a random subject effect. The two models were compared using the anova( ) function in R, using the likelihood ratio test. Significant p-values were then adjusted for a false discovery rate (FDR) using the Benjamini-Hochberg procedure (Benjamin and Hochberg (1995), the entire contents of which are herein incorporated by reference). Probe sets were identified as significantly differentially expressed for age when the FDR p-value was less than 0.05.

Of the 54,675 transcripts on the array, 9,286 showed significant expression changes over time (i.e., —a p-value less than 0.05). Key biomarkers of interest of this study, including EGF, IL8, TLR, and PAF were all detected in the saliva and found to significantly change over time. Related genes including the IL10 receptor and EGF receptor were also identified. These results confirmed that single genes could be analyzed using gene expression array technology.

In addition to single gene analysis, data were used to analyze genes on a global level and to shed light on possible interactions between gene products. Based on calculated T-scores, the significant gene list was divided into those showing a trend towards decreased expression over time (negative T score; n=3522), and those showing a trend in increased expression over time (positive T score; n=5764). Each respective gene list was then entered into Ingenuity® for a formal, comprehensive analysis. Ingenuity® is an integrated commercially available database that allows researchers to search, explore, visualize, and analyze biological and chemical findings related to genes, proteins, and small molecules (e.g., drugs). Ingenuity® assesses how individual genes within a group relate to one another and calculates statistically over-represented systems within such a described list. Significant over-represented networks are group into one or more categories: Physiological System Development and Function, Molecular and Cellular Functions, Disease and Disorders, Toxicity Pathways, and Canonical Pathways. The top 5 up-regulated and down-regulated physiological development systems identified with Ingenuity® are depicted in Tables 2 and 3, respectively.

TABLE 2
Top 5 up-regulated physiological development systems

| Physiological | Approximate | # of |
|---|---|---|
| System | P-values | Genes |
| Behavior | from ~1.2 × 10⁻¹¹ to ~1.5 × 10⁻² | 170 |
| Nervous System | from ~1.8 × 10⁻⁹ to ~1.7 × 10⁻² | 407 |
| Development and | | |
| Function | | |
| Tissue Development | from ~4.6 × 10⁻⁷ to ~1.7 × 10⁻² | 226 |
| Organ Development | from ~6.0 × 10⁻⁶ to ~1.6 × 10⁻² | 227 |
| Digestive System | from ~1.2 × 10⁻⁵ to ~1.3 × 10⁻² | 49 |
| Development and | | |
| Function | | |

TABLE 3
Top 5 down-regulated physiological development systems

| Physiological | Approximate | # of |
|---|---|---|
| System | P-values | Genes |
| Embryonic development | from ~7.2 × 10⁻¹⁴ to ~1.2 × 10⁻³ | 128 |
| Connective Tissue | from ~4.8 × 10⁻⁸ to ~2.3 × 10⁻³ | 147 |
| Development and | | |
| Function | | |
| Hematological | from ~1.3 × 10⁻⁷ to ~2.3 × 10⁻³ | 225 |
| System Development | | |
| and Function | | |
| Hematopoiesis | from ~1.3 × 10⁻⁷ to ~9.2 × 10⁻⁴ | 122 |
| Organismal Survival | from ~6.5 × 10⁻⁷ to ~2.1 × 10⁻³ | 173 |

As can be seen from Tables 2 and 3, neonatal salivary genomic analysis can indeed provide a window into the premature infant's gastrointestinal development and neurodevelopment as an infant learns to orally feed. Furthermore, it was unexpectedly discovered that genomic analysis of neonatal saliva provides a picture of overall global development of a developing premature infant.

Among the over 9,000 genes that were differentially expressed during the course of infant development (that is, during the days after birth of the premature infant), genes with the most highly significant (p<0.001) expression differences were identified. These included both upregulated and downregulated genes and are discussed further below.

### Highly Significantly Upregulated Genes

A number of highly significantly upregulated genes are involved in development of the digestive system and/or in digestion. Several of these upregulated genes have functions relating to feeding. For example, neuropeptide Y receptor Y1 (NPY1R) was found to be upregulated over time. Neuropeptide Y is one of the most abundant neuropeptides in the mammalian system, with a diverse range of important physiologic functions, including food intake. Other upregulated genes include Leptin Receptor (LEPR), a receptor to an adipocyte-specific hormone that regulates adipose tissue mass through hypothalamic effects on satiety and energy; growth hormone secretagogue receptor (GHSR), which may play a role in energy homeostasis and regulation of body weight; and prostaglandin E receptor 3 (subtype EP3) (PTGER 3), which may have many biological functions involving digestion, the nervous system, kidney reabsorption, and uterine contraction activities.

Highly significantly upregulated genes involved in digestion also featured genes involved in feeding behavior, such as hypocretin (orexin) receptor 2 (HCRTR2), a G-protein coupled receptor involved in the regulation of feeding behavior. Orexins are believed to be primarily involved in stimulation of food intake, wakefulness, and energy expenditure. Galanin receptor 3 (GALR3), a neuropeptide that modulates a variety of physiologic processes including cognition, sensory/pain processing, hormone secretion, and feeding behavior, was also found to be upregulated. Lactalbumin alpha (LALBA) and glucagon (GCG) were also upregulated. Alpha lactalbumin is a principal protein of milk and forms the regulatory subunit of the lactose synthase heterodimer that enables production of lactose by transferring galactose moieties to glucose. Glucagon is a pancreatic hormone that counteracts the glucose-lowering action of insulin by stimulating glycogenolysis and gluconeogenesis.

Additionally, there were 407 gene transcripts involved in nervous system development whose up-regulation over time was highly significant. These gene transcripts were involved in a broad range of aspects of nervous system development, including development of neurons, nerves, the central nervous system, and nervous tissue; formation of oligodendrocytes and neuroglia; growth of neurites; and myelination. One particular nerve was highlighted among these genes: the trigeminal nerve (CN V). Genes that were upregulated over time included some that were involved in three specific functions or aspects associated with trigeminal nerve's: development of trigeminal ganglion nerves, quantity of trigeminal ganglion neurons, and survival of trigeminal ganglion neurons. The trigeminal nerve transmits somatosensory information (such as touch and pain) from the face and head and innervates muscles involved in chewing. Genes involved in olfactory system development (including development of olfactory bulb and of olfactory receptor neurons) were also upregulated in a highly significant manner.

Feeding associated genes that displayed highly significant upregulation over time included receptors involved in regulating food consumption. These genes included melanin-concentrating hormone receptor 1 (MCHR1), which is likely involved in neuronal regulation of food consumption; prostaglandin E receptor 3 (PTGER3),; a receptor that has many biological functions including digestion, nervous system, kidney reabsorption, and uterine contraction activities; and cholecytokinin A receptor (CCKAR), a major physiologic mediator of pancreatic enzyme secretion and smooth muscle contraction of the gallbladder and stomach. In the central and peripheral nervous system, cholecytokinin A receptor regulates satiety and the release of beta-endorphin and dopamine.

Genes involved in sniffing were also found to be highly significantly upregulated and included odorant binding protein 2B (OBP2B); transient receptor potential cation channel, subfamily V, member 1 (TRPV1); and taste receptor, type 2, member 1 (TAS2R1). TRPV1 encodes a receptor for capsaicin, an ingredient that elicits a sensation of burning pain. The receptor conveys information about noxious stimuli to the central nervous system and is also activated by increases in temperature in the noxious range, which may indicate that it functions as a transducer of painful thermal stimuli in vivo. TAS2R1 encodes a member of a family of candidate taste receptors that belong to the G protein coupled receptor superfamily and that are specifically expressed by taste receptor cells of the tongue and palate epithelia.

Several genes involved in respiratory development were also highly significantly upregulated. These genes include surfactant protein B (SFTPB), an amphipathic surfactant protein essential for lung function and homeostasis after birth; cystic fibrosis transmembrane conductance regulator (CFTR), a chloride channel that controls regulation of other transport pathways; fibroblast growth factors (FGF) 1, 2, 7, 10, 18, which have broad mitogenic and cell survival activities and are involved in a variety of biological processes (including embryonic development, cell growth, morphogenesis, tissue repair, tumor growth, and invasion); and fibroblast growth receptor 2 (FGFR2), which has been implicated in diverse biological processes such as limb and nervous system development, wound healing, and tumor growth.

### Highly Significantly Downregulated Genes

A number of highly significantly downregulated genes are involved in embryonic development. One such gene is carcinoembryonic antigen-related cell adhesion molecule 1 (biliary glycoprotein) (CEACAM1), a cell-cell adhesion molecule detected on leukocytes, epithelia, and endothelia. CEACAM1 is involved in the arrangement of tissue three-dimensional structure, angiogenesis, apoptosis, tumor suppression, metastasis, and modulation of innate and adaptive immune responses. Another embryonic development gene identified as being downregulated is V-raf murine sarcoma viral oncogene homolog B1 (BRAF), which plays a role in regulating the MAP kinase/ERK signaling pathway, which affects cell division, differentiation, and secretion. Mutations in BRAF are associated with cardiofaciocutaneous syndrome. Other down-regulated genes included amino-terminal enhancer of split (AES), which is involved in neurogenesis during embryonic development; E1A binding protein p300 (EP300), which has been identified as a co-activator of HIF1A (hypoxia-inducible factor 1 alpha) and plays a role in stimulating hypoxia induced genes such as VEGF; Fas (TNF receptor superfamily member 6) (FAS), a receptor that contains a death domain, has been shown to play a central role in the physiological regulation of programmed cell death, and has been implicated in the pathogenesis of various malignancies and diseases of the immune system; and Fas (TNFRSF6)-associated via death domain (FADD), an adaptor molecule that interacts with various cell surface receptors and mediates cell apoptotic signals. FADD knockout studies in mice suggest the importance of FADD in early T cell development.

Another set of highly significantly downregulated genes are involved in organismal survival. One such gene is cyclin-dependent kinase inhibitor 2A (melanoma, p16, inhibits CDK4) (CDKN2A), a stabilizer of the tumor suppressor protein p53. CDKN2A is frequently mutated or deleted in a wide variety of tumors and is known to be an important tumor suppressor gene. Other downregulated genes include glycogen synthase kinase 3 Beta (GSK3B), a phosphorylating and inactivating glycogen synthase that is involved in energy metabolism, neuronal cell development, and body pattern formation; protein kinase, cAMP-dependent, regulatory, type 1, alpha (tissue specific extinguisher 1) (PRKAR1A), a tissue-specific extinguisher that down-regulates expression of seven liver genes in hepatoma-fibroblast hybrids; signal transducer and activator of transcription 5B (STAT5B), which mediates signal transduction triggered by various cell ligands (such as IL2, IL4, CSF1, and different growth hormones) and is involved in diverse processes (such as TCR signaling apoptosis, adult mammary gland development, and sexual dimorphism of liver gene expression); aryl hydrocarbon receptor nuclear translocator (ARNT), which is involved in induction of several enzymes that participate in xenobiotic metabolism and is identified as the beta subunit of a heterodimeric transcription factor (hypoxia-inducible factor 1; and insulin receptor (INSR), which together with its ligand insulin stimulates glucose uptake.

These experiments identified genes involved in neonatal development of premature infants, including genes involved in feeding. Furthermore, these results confirm that in addition to allowing analysis of a single gene or protein of interest, microarray technology also facilitates analysis of interactions between multiple related genes during normal postnatal development and/or in the presence of disease.

### Examples 5-7

### Profiling to Examine Readiness to Feed, Tolerance of Feeding, and Necrotizing Enterocolitis (NEC)

In these Examples, neonatal salivary genomic expression profiles are obtained and used to provide novel and informative data regarding development and physiological conditions related to feeding and/or NEC. Experiments described in these Examples are expected to identify certain genes and/or sets of genes as biomarkers that can be used to make certain determinations. These determinations may include, among other things, whether a neonate is ready to feed, a neonate's tolerance of feeds, and/or whether a neonate is at risk for developing, has developed, or is in a particular stage of, etc., NEC.

### Target Population for Enrollment

Neonates born between 28 and 34 weeks' gestation without known anomalies or genetic diseases are targeted for enrollment. Such neonates have an increased likelihood of developing feeding intolerance and NEC due to their prematurity at birth. Several factors guide the decision to target neonates born between 28 and 34 weeks' gestation. First, during preliminary data acquisition, salivary samples were most successfully obtained from neonates who weighed ≧1,000 g. To ensure continued success, neonates born at ≧28 weeks in gestational age, who have an expected average birth weight of approximately 1,000 g, are targeted. Second, while the inventors acknowledge that neonates born before 28 weeks' gestation likely have a higher incidence of NEC, such neonates also likely have a higher incidence of many other complications of prematurity resulting in elevated co-morbidity and mortality rates. In the interest of limiting confounding variables associated with such elevated co-morbidity and mortality rates, neonates born before 28 weeks' gestation are excluded from these studies. It is intended in these studies to capture neonates as they begin orally feeding. At the Floating Hospital for Children's NICU, where these studies are conducted, it is the general practice to introduce oral feeding at ≧34 weeks' gestation. Targeting neonates born up to 34 weeks should maximize the ability to capture the appropriate study subjects for this example.

### Acquisition of Saliva and Selection of Neonates for Gene Expression Microarray Experiments

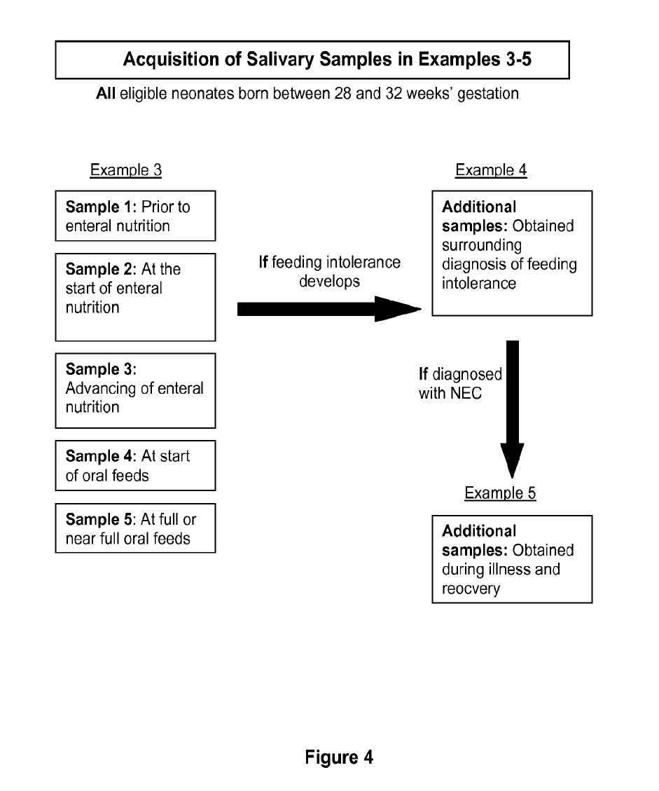

Saliva is obtained serially for all enrolled neonates throughout their hospitalizations. Because oral suctioning of neonates is part of routine neonatal care in the NICU, and the obtainment of saliva samples is expected to pose no threat to the neonates. A timeline for saliva acquisition for experiments described in these Examples is depicted in FIG. 4.

Samples are intentionally acquired repetitively in these studies for at least two reasons. First, as stated previously, neonates enrolled in these studies may develop other complications of prematurity. Collecting serial samples from the same neonate over time affords a possible way to control for such variations. Second, expression levels of genes of interest may fluctuate. While some genes (such as, for example, housekeeping genes) may show little variation from day to day or week to week, other genes (such as, for example, neurodevelopmental genes and genes involved in inflammation) are often dynamically expressed. Sampling saliva from the same neonates serially may allow pinpointing specific genes involved in normal physiologic and/or in various pathological processes relevant to developmental pathways in the preterm neonate.

Salivary RNA from each neonate in these studies are obtained and stored. The decision to perform gene expression microarray experiments on particular neonates are made retrospectively (i.e., after clinical outcomes of the neonates are known). Neonates are selected for microarray expression analysis if complete sets of adequate salivary samples were obtained from them and if the neonates meet relevant clinical criteria for appropriate comparisons for each particular study. Salivary samples from neonates not selected for microarray expression analysis are appropriately processed and stored for possible subsequent use in developing a larger genomic expression data panel, a long range goal of this work.

### Statistical Analyses

It has been estimated that at least five gene expression microarray analyses may be needed to provide sufficient power for the intended analyses in these Examples. Therefore, for each Example, salivary samples from no fewer than 5 neonates are considered in each arm of the analysis.

Microarray data analyses are performed in R using the Affy and Multtest packages in Bioconductor (Gentleman R. C. et al. 2004). Array data are normalized using the quantile normalization method. ANOVAs are performed and p-values will be adjusted for multiple testing using the Benjamini-Hochberg false discovery rate approach (Benjamini and Hochberg (1995)). Candidate biomarkers are selected if their adjusted p-values are less than 0.05. Analyses of sets of genes in known pathways are also performed using Gene Set Enrichment Analysis (GSEA). (Romero and Tromp (2006), the entire contents of which are herein incorporated by reference in their entirety.) This analytical method can identify subtle but consistent gene expression changes in previously defined pathways. Once lists of genes with statistically significant expression differences are generated for each comparison, information (e.g., functional roles and expression patterns) about each gene in the list from publically available databases (e.g., OMIM, http://www.ncbi.nlm.nih.gov/sites/entrez?db=OMIM; PubMed, https://www.ncbi.nlm.nih.gov/sites/entrez; NetAffx, http://www.affymetrix.com/analysis/index.affx; UniGene, http://www.ncbi.nlm.nih.gov.sites/entrez?db=unigene; etc.), as well as commercially available databases, (e.g. Ingenuity), are manually reviewed to determine the potential role of each gene in development related to feeding and physiological readiness to feed.

### Example 5

### Identification of Genes that May be Used as Biomarkers of a Neonate's Readiness to Feed

In this Example, neonatal salivary genomic expression profiles are obtained and used to provide novel and informative data regarding a neonate's readiness to feed. Saliva samples are collected from enrolled neonates at particular timepoints: prior to the initiation of enteral feeding, following introduction of enteral feeds, and during the learning process of oral feeding. Expression profiles of developmental genes are chronicled in the developing preterm neonate by analyzing samples from such timepoints. Experiments described in this Example may identify mucosal, mesenchymal, and neurodevelopmental genes whose transcripts are expressed as neonates begin to orally feed. Such genes may be useful as biomarkers to determine a neonate's readiness to feed.

After parental consent and Health Insurance Portability and Accountability Act (HIPAA) authorization, each neonate and all corresponding salivary samples are assigned a code known only to the Principal Investigator, lab manager, and NRN research nurse. Salivary samples are obtained at four time points of interest: 1) prior to the initiation of enteral feeds; 2) following the introduction of enteral feeds once a neonate reaches half volume of full feeds; 3) at the introduction of oral feeding; and 4) at full oral feeds. For each time point, the oropharynx of the neonate is gently suctioned to collect approximately 100 μL to approximately 200 μL of saliva just prior to a feed to reduce the risk of contamination from formula or breast milk. Salivary samples are immediately stabilized with Qiagen™ RNAprotect Saliva Reagent. Salivary RNA extraction are subsequently performed with the commercially available PaxGene RNEasy® Protect Saliva kit. Extracted salivary RNA is stored at −80° C. until future analysis.

For microarray analysis, stored extracted salivary RNA is amplified, biotinylated, and fragmented with the Nugen™ Pico Amplification and Biotinylation and Fragmenting kits. Quality and quantity of amplified salivary samples is assessed with the Agilent™ BioAnalyzer 2100. Approximately 5 μg of amplified salivary mRNA is then hybridized onto the Affymetrix™ HGU133 Plus 2.0 array. Arrays are washed, stained, and scanned. Bioinformatic analyses is performed on the microarray data to identify genes whose expression levels differ among the time points of saliva collection in this study.

Expression of genes that are identified as differentially expressed and that are believed to play a key role in the development of normal oral feeding patterns is quantified further by RT-PCR. RT-PCR is performed on remaining, stored, unamplified salivary samples by TaqMan™ amplification on an Applied Biosystem™ 7900 Sequence Detection System.

Gene expression levels of first, second, third, and fourth samples from each neonate in this study are compared using ANOVAs. The experiments in this Example may identify genes that are consistently changing between at least one pair of these groups of samples, and ultimately identify key mesenchymal genes necessary for the proper processing of enteral nutrition. Key neurodevelopmental genes necessary for successful oral feeding and gut motility are also expected to be identified in this study.

### Example 6

### Identification of Genes that May be Used as Biomarkers of Feeding Intolerance

In this Example, neonatal salivary genomic expression profiles are obtained and used to provide novel and informative data regarding the pathophysiology of feeding intolerance. Data from neonates who demonstrate feeding intolerance will be compared against data from those who do not. Without wishing to be bound by any particular theory, it is contemplated that longitudinal genomic analyses of feeding-intolerant neonates will demonstrate upregulation of inflammatory (e.g., cytokines) and/or allergic (e.g., IgE) markers and/or disregulation of essential digestive enzymes. Such differentially or disregulated genes may potentially be used as biomarkers to differentiate between true pathology and more benign conditions. For example, it may be possible to distinguish, using such biomarkers, neonates suffering from a true formula allergy from neonates who may have an evolving pathological condition. Identification of genes involved in the pathophysiology of feeding intolerance may also allow prospective identification of some neonates who will subsequently develop NEC.

In this Example, specific comparisons are also made between neonates who demonstrate feeding intolerance who are exclusively breastfed and those who are exclusively formula-fed. It is contemplated, without wishing to be bound by any particular theory, that salivary expression profiles between these cohorts of neonates should be different, and that comparative analysis allows identification of biomarkers within the breastfeeding group that may explain the presumed protective effect against the development of NEC conferred upon premature breastfeeding neonates.

Samples are collected from neonates chosen for this study as described in the above “Target population for enrollment” section. In this Example, additional samples are collected from neonates who demonstrated feeding intolerance upon the introduction of enteral feeding. For the purposes of this Example, neonates are classified as feeding intolerant if the neonate has one or more of the following conditions: a) persistently heme positive stools without evidence of anal fissure or abrasions; b) abdominal distension warranting discontinuation of feeds or formula change; c) formula residuals representing 25% of initial feeds for at least 2 feeds within a 24 hour period; and d) inability to advance to or maintain full enteral feeds.

Statistical comparisons are made between gestational age-matched neonates who had no difficulty feeding and those who developed feeding intolerance as previously described. For comparative analysis, neonates in each group must have a complete set of adequate salivary RNA. For each group, two-way ANOVAs will be performed on salivary genomic profiles on all available preceding time points. It is expected that by performing comparisons between groups of saliva collected over time, it is possible to identify discrepancies between neonates with feeding intolerance and those without for a particular time point. Additionally or alternatively, it may be possible to identify genetic markers of feeding intolerance whose expression change over time. Experiments and analyses described this Example may yield predictive markers that can be used to identify neonates at risk for developing feeding complications.

### Example 7

### Identification of Genes that May be Used as Biomarkers of Necrotizing Enterocolitis (NEC)

In this Example, neonatal salivary genomic expression profiles are generated from samples obtained during the acute and convalescent stages of NEC. Such expression profiles may be used to provide novel and informative data regarding the pathophysiology of NEC. Comprehensive genomic information generated by the experiments in this Example, combined with information obtained from Example 6 (which may be useful for prospectively identifying neonates at risk for developing NEC) may highlight specific genes involved in the pathophysiology of NEC. Such genes may help elucidate mechanisms of NEC pathophysiology and serve as targets for future studies and therapy.

Samples are collected from neonates chosen for this study as described in the above “Target population for enrollment” section. In this Example, additional samples are collected from neonates diagnosed with NEC based on clinical and radiographic or surgical findings. Samples are obtained immediately following diagnosis, and then 1-2 times per week additionally during the neonate's convalescence.

On average, there are approximately four cases (ranging from 2 to 8 cases) of NEC diagnosed per year in the Floating Hospital for Children's NICU. (These numbers are based on records from 1997-2006 at the NICU.) Because of the limited numbers of expected neonates with NEC, any neonate diagnosed with NEC is incorporated into this study. Saliva sample acquisition, RNA extraction and processing, microarray experimental design, and statistical analyses are performed as described previously in the “Acquisition of saliva and selection of neonates for gene expression microarray experiments,” and “Statistical analyses” sections above and as described in Example 5. ANOVAs are performed between neonates who developed NEC and those who did not for all available time points.

All literature and similar material cited in this application, including, patents, patent applications, articles, books, treatises, dissertations and web pages, regardless of the format of such literature and similar materials, are expressly incorporated by reference in their entirety. In the event that one or more of the incorporated literature and similar materials differs from or contradicts this application, including defined terms, term usage, described techniques, or the like, this application controls.

The section headings used herein are for organizational purposes only and are not to be construed as limiting the subject matter described in any way.

### REFERENCES

1. Adams et al., “Complementary DNA sequencing: expressed sequence tags and human genome project.” Science. 1991, 252: 1651-1656
2. Aikens et al., “Solid-state imagers for microscopy.” Meth. Cell Biol. 1989, 29: 291-313
3. Alberts et al., “Molecular Biology of the Cell.” 1994 (3rd Ed.), Garland Publishing, Inc.: New York, N.Y.
4. Anderson (Ed.), “Nucleic Acid Hybridization.” 1999, Springer Verlag: New York, N.Y.
5. Anfinsen et al., “Studies on the gross structure, cross-linkages, and terminal sequences in ribonuclease.” J. Biol. Chem. 1954, 207: 201-210
6. Ausubel (Ed.), “Short Protocols in Molecular Biology.” 2002, 5th Ed., John Wiley & Sons;
7. Barringer et al., “Blunt-end and single-strand ligations by Escherichia coli ligase: influence on an in vitro amplification scheme.” Gene. 1990, 89: 117-122
8. Bassett et al., “Gene expression informatics—it's all in your mine.” Nat. Genet. 1999, 21: 51-55
9. Bates et al. “Ime4: Linear mixed-effects models using S4 classes. R package version 0.999375-28.” http://lme4.r-forge.r-project.org.
10. Bayer et al., “The use of the avidin-biotin complex as a tool in molecular biology.” Methods of Biochem. Analysis. 1980, 26: 1-45
11. Becker-Andre and Hahlbrock, “Absolute mRNA quantification using the polymerase chain reaction (PCR). A novel approach by a PCR aided transcript titration assay (PATTY).” Nucl. Acids Res. 1989, 17: 9437-9346
12. Blackburn et al. “Ribonuclease inhibitor from human placenta. Purification and properties.” J. Biol. Chem. 1977, 252: 5904-5910
13. Benjamini and Hochberg. “Controlling the false discovery rate: a practical and powerful approach to multiple testing.” J R Stat Soc (Ser A) 1995 B75:290-300
14. Bravo et al., “A versatile negative-staining ribonuclease zymogram.” Anal. Biochem. 1994, 219: 82-86
15. Brazma and Vilo, “Gene expression data analysis.” FEBS Lett. 2000, 480: 17-24
16. Brison et al., “General method for cloning amplified DNA by differential screening with genomic probes.” Mol. Cell. Biol. 1982, 2: 578-587
17. Brigati et al., “Detection of viral genomes in cultured cells and paraffin-embedded tissue sections using biotin-labeled hybridization probes.” Virol. 1983, 126: 32-50
18. Britten et al., “Analysis of repeating DNA sequences by reassociation.” Methods in Enzymol., 1974, 29: 363-418
19. Broker et al., “Electron microscopic visualization of tRNA genes with ferritin-avidin: biotin labels.” Nucl. Acids Res. 1978, 5: 363-384
20. Burg et al., “Single molecule detection of RNA reporter probes by amplification with Q beta replicase.”Mol. Cell. Probes. 1996, 10: 257-271
21. Butte, “The use and analysis of microarray data.” Nat. Rev. Drug Discov. 2002, 1: 951-960
22. Chen et al., “Profiling Expression Patterns and Isolating Differentially Expressed Genes by cDNA Microarray System with Colorimetry Detection.” Genomics, 1998, 51: 313-324
23. Chen et al., “4,4-Difluoro-4-bora-3a,4a-diaza-s-indacene (BODIPY) Dyes Modified for Extended Conjugation and Restricted Bond Rotations.” J. Org. Chem. 2000, 65: 2900-2906
24. Chen et al., “Probing the cathepsin D using a BODIPY FL-pepstatin A: applications in fluorescence polarization and microscopy.” J. Biochem. Biophys. Methods, 2000, 42: 137-151
25. Cheung et al., “Making and reading microarrays.” Nature Genet. 1999, 21: 15-19
26. Chomczynski et al., “Single-step method of RNA isolation by acid guanidinium thiocyanate-phenol-chloroform extraction.” Anal. Biochem. 1987, 162: 156-159
27. Chiang et al., “Visualizing associations between genome sequences and gene expression data using genome-mean expression profiles.” Bioinformatics, 2001, 17: S49-S55
28. Chirgwin et al., Biochem. 1979, 18: 5294-529
29. Connoly and P. Rider, “Chemical synthesis of oligonucleotides containing a free sulphydryl group and subsequent attachment of thiol specific probes.” Nucl. Acids. Res. 1985, 13: 4485-4502
30. Dewey, “From microarrays to networks: mining expression time series.” Drug Discov. Today, 2002, 7: S170-S175
31. Divane et al., “Rapid prenatal diagnosis of aneuploidy from uncultured amniotic fluid cells using 5 colour fluorescence in situ hybridization.” Prenat. Diagn. 1994, 14: 1061-1069
32. Duggan et al., “Expression profiling using cDNA microarrays.” Nat. Genet. 1999, 21: 10-14
33. Ekins and Chu, “Microarrays: their origins and applications.” Trends in Biotech. 1999, 17: 217-218
34. Gautier et al., “Affy-analysis of Affymetrix GeneChip data at the probe level.” Bioinformatics. 2004 20:307-315.
35. Gentleman et al. “Bioconductor: open software development for computational biology and bioinformatics.” Genome Biol. 2004 5: R(80).
36. Gilliland et al., “Analysis of cytokine mRNA and DNA: detection and quantitation by competitive polymerase chain reaction.” Proc. Natl. Acad. Sci. 1990, 87: 2725-2729
37. Glisin et al., “Ribonucleic acid isolated by cesium chloride centrifugation.” Biochem. 1974, 13: 2633-2637
38. Greijer et al., “Quantitative competitive NASBA for measuring mRNA expression levels of the immediate early 1, late pp 67, and immune evasion genes US3, US6, and US11 in cells infected with human cytomegalovirus.” J. Virol. Methods. 2001, 96: 133-147
39. Greiner-Stoeffele et al., “A General Ribonuclease Assay Using Methylene Blue.” Anal. Biochem. 1996, 240: 24-28
40. Grunert et al., “Trp59 to Tyr substitution enhances the catalytic activity of RNase T1 and of the Tyr to Trp variants in positions 24, 42 and 45.” Protein Eng. 1993, 6: 739-744
41. Guatelli et al., “Isothermal, in vitro amplification of nucleic acids by a multienzyme reaction modeled after retroviral replication.” Proc. Natl. Acad. Sci. 1990, 87: 1874-1878
42. Hamaguchi et al., “Aptamer Beacons for the Direct Detection of Proteins.” Anal. Biochem. 2001, 294: 126-131
43. Haugland, “Molecular Probes: Handbook of Fluorescent Probes and Research Chemicals 1992-1994”, 5th Ed., 1994, Molecular Probes, Inc.
44. Hess et al., “Microarrays: Handling the deluge of data and extracting reliable information.” Trends Biotechnol. 2001, 19: 463-468
45. Hiraoka et al., “The Use of a Charge-Coupled Device for Quantitative Optical Microscopy of Biological Structures.” Science, 1987, 238: 36-41
46. Hopman et al., “Mercurated nucleic acid probes, a new principle for non-radioactive in situ hybridization.” Exp. Cell Res. 1987, 169: 357-368
47. Jalal et al., “Prenatal detection of aneuploidy by directly labeled multicolored probes and interphase fluorescence in situ hybridization.” Mayo Clin. Proc. 1998, 73: 132-137
48. Kerr et al., “Analysis of Variance for Gene Expression Microarray Data.” J. Comput. Biol. 2000, 7: 819-837
49. Marcotte and Date, “Exploiting big biology: integrating large-scale biological data for function inference.” Brief Bioinform. 2001, 2: 363-374
50. Ikehara et al., “Inquiries into the structure-function relationship of ribonuclease T1 using chemically synthesized coding sequences.” Proc. Natl. Acad. Sci. USA, 1986, 83: 4695-4699
51. Innis (Ed.), “PCR Protocols: A Guide to Methods and Applications.” Academic Press: New York, 1990
52. Innis (Ed.), “PCR Strategies.” Academic Press: New York, 1995
53. Joos et al., “Mapping and chromosome analysis: the potential of fluorescence in situ hybridization.” J. Biotechnol. 1994, 35: 135-153
54. Kaboev et al., “PCR hot start using primers with the structure of molecular beacons (hairpin-like structure).” Nucl. Acids Res. 2000, 28: E94
55. Kievits et al., “NASBA™ isothermal enzymatic in vitro nucleic acid amplification optimized for the diagnosis of HIV-1 infection.” J. Virol. Methods, 1991, 35: 273-286
56. Kimmel and Berger, “Preparation of cDNA and the generation of cDNA libraries: Overview.” Methods in Enzymol. 1987, 152: 307-316
57. Korn et al., “Analysis of the RNase T1 Mediated Cleavage of an Immobilized Gapped Heteroduplex via Fluorescence Correlation Spectroscopy.” Biol. Chem. 2000, 381: 259-263
58. Kricka, “Stains, labels and detection strategies for nucleic acids assays.” Ann. Clin. Biochem. 2002, 39: 114-129
59. Kwoh et al., “Transcription-based amplification system and detection of amplified human immunodeficiency virus type 1 with a bead-based sandwich hybridization format.” Proc. Natl. Acad. Sci. 1989, 86: 1173-1177
60. Landegren et al., “A ligase-mediated gene detection technique.” Science, 1988, 241: 1077-1080
61. Landegent et al., “2-acetylaminofluorine-modified probes for the hybridocytochemical detection of specific nucleic acid sequences.” Exp. Cell Res. 1984, 15: 61-72
62. Langer et al., “Enzymatic synthesis of biotin-labeled polynucleotides: novel nucleic acid affinity probes.” Proc. Natl. Acad. Sci., 1981, 78: 6633-6637
63. Liang and Pardee, “Differential display of eukaryotic messenger RNA by means of the polymerase chain reaction.” Science, 1992, 257: 967-971
64. Livak et al., “Oligonucleotides with Fluorescent Dyes at Opposite Ends Provide a Quenched Probe System Useful for Detecting PC Product and Nucleic Acid Hybridization.” PCR Methods Appl. 1995, 4: 357-362
65. Lockhart and Winzeler, “Genomics, gene expression and DNA arrays.” Nature, 2000, 405: 827 836
66. Mansfield et al., “Nucleic acid detection using non-radioactive labelling methods.” Mol. Cell. Probes. 1995, 9: 145-156
67. Marshall and Hodgson, “DNA chips: an array of possibilities.” Nature Biotech. 1998, 16: 27-31
68. McClelland et al., “Arbitrary primed PCR fingerprinting of RNA applied to mapping differentially expressed genes.” EXS, 1993, 67: 103-115
69. Neves et al., “Novel method for covalent fluorescent labeling of plasmid DNA that maintains structural integrity of the plasmid.” Bioconjugate Chem. 2000, 11: 51-55
70. Poddar and Le, “Bordetella pertussis detection by spectrofluorometry using polymerase chain reaction (PCR) and a molecular beacon probe.” Mol. Cell. Probes, 2001, 15: 161-167
71. Quaas et al., “Indicator plates for rapid detection of ribonuclease T1 secreting Escherichia coli clones.” Nucl. Acids Res. 1989, 17: 3318
72. R Development Core Team. “R: A language and environment for statistical computing. R Foundation for Statistical Computing.” 2008 Vienna, Austria. ISBN 3-900051-07-0, URL http://www.R-project.org.
73. Ramsay, “DNA chips: state-of-the art.” Nature Biotech. 1998, 16: 40-4
74. Richardson et al., “Biotin and fluorescent labeling of RNA using T4 RNA ligase.” Nucl. Acids Res. 1983, 11: 6167-6184
75. Sambrook et al., “Molecular Cloning: A Laboratory Manual”, 1989, 2nd Ed., Cold Spring Harbour Laboratory Press: New York)
76. Schena et al., “Quantitative monitoring of gene expression patterns with a complementary DNA microarray.” Science, 1995, 270: 467-470
77. Schena et al., “Parallel human genome analysis: microarray-based expression monitoring of 1000 genes.” Proc. Natl. Acad. Sci. 1996, 93: 10614-10619
78. Sebestyen et al., “DNA vector chemistry: the covalent attachment of signal peptides to plasmid DNA.” Nat. Biotechnol. 1998, 16: 568-576
79. Smith et al., “Detection of Mycobacterium tuberculosis directly from sputum by using a prototype automated Q-beta replicase assay.” J. Clin. Microbiol. 1997, 35(6): 1477-1491
80. Smith et al., “The synthesis of oligonucleotides containing an aliphatic amino group at the 5′ terminus: synthesis of fluorescent DNA primers for use in DNA sequence analysis.” Nucl. Acids Res. 1985, 13: 2399-2412
81. Sokol et al., “Real time detection of DNA RNA hybridization in living cells.” Proc. Natl. Acad. Sci., 1998, 95: 11538-11543
82. Stern and Newton, “Isolation of plant mitochondrial RNA.” Methods in Enzymol. 1986, 118: 488
83. Su et al., “High-throughput RT-PCR analysis of multiple transcripts using a microplate RNA isolation procedure.” BioTechniques, 1997, 22: 1107-1113
84. Tchen et al., “Chemically modified nucleic acids as immunodetectable probes in hybridization experiments.” Proc. Natl Acad. Sci. 1984, 81: 3466-3470
85. Tamames et al., “Bioinformatics methods for the analysis of expression arrays: data clustering and information extraction.” J. Biotechnol. 2002, 98: 269-283
86. Temsamani and Agrawal, “Enzymatic labeling of nucleic acids.” Mol. Biotechnol. 1996, 5: 223-232
87. van Gijlswijk et al., “Universal Linkage System: versatile nucleic acid labeling technique.” Expert Rev. Mol. Diagn. 2001, 1: 81-91
88. Velculescu et al., “Serial analysis of gene expression.” Science, 1995, 270: 484-487
89. Wang et al., “Quantitation of mRNA by the polymerase chain reaction.” Proc. Natl. Acad. Sci. 1989, 86: 9717-9721
90. Watson et al., “Technology for microarray analysis of gene expression.” Curr. Opin. Biotechnol. 1998, 9: 609-614
91. Welsh et al., “Arbitrarily primed PCR fingerprinting of RNA.” Nucl. Acids Res. 1992, 20: 4965-497
92. Wiegant et al., “ULS: a versatile method of labeling nucleic acids for FISH based on a monofunctional reaction of cisplatin derivatives with guanine moieties.” Cytogenet. Cell. Genet. 1999, 87: 47-52
93. Wilfinger et al., “Effect of pH and ionic strength on the spectrophotometric assessment of nucleic acid purity.” Biotechniques. 1997, 22: 474-481
94. Weinstein et al., “The bioinformatics of microarray gene expression profiling.” Cytometry. 2002, 47: 46-49
95. Xiang et al., “Microarray expression profiling: analysis and applications.” Curr. Opin. Drug Discov. Devel. 2003, 6: 384-395
96. Yamamoto et al., “Molecular beacon aptamer fluoresces in the presence of Tat protein of HIV-1.” Genes Cells. 2000, 5: 389-396
97. Zhang et al., “Gene expression profiles in normal and cancer cells.” Science. 1997, 276: 1268-1272

### OTHER EMBODIMENTS

Other embodiments of the invention will be apparent to those skilled in the art from a consideration of the specification or practice of the invention disclosed herein. It is intended that the specification and examples be considered as exemplary only, with the true scope of the invention being indicated by the following claims.

What is claimed is:

1. A method for detecting or identifying genes involved in a condition or disease affecting neonates comprising steps of:
providing a test sample of saliva RNA obtained from a neonate suffering from or diagnosed with a condition;
subjecting the test sample of saliva RNA to an analysis, wherein the analysis comprises hybridizing the RNA to one or more oligonucleotide probes, such that one or more genes that are differentially regulated in the sample as compared to a control sample is/are identified, wherein the control sample comprises saliva RNA obtained from a neonate that is not suffering from or diagnosed with the condition; and
determining that the one or more differentially regulated genes are involved in the condition or disease.

2. The method of claim 1, wherein the condition is selected from the group consisting of necrotizing enterocolitis, respiratory distress syndrome, bronchopulmonary dysplasia, sepsis, and combinations thereof.

3. The method of claim 2, wherein the condition is necrotizing enterocolitis.

4. A method for detecting or identifying genes involved in neonatal development comprising steps of:
providing a test sample of saliva RNA obtained from a neonate;
subjecting the test sample of saliva RNA to an analysis, wherein the analysis comprises hybridizing the RNA to one or more oligonucleotide probes, such that one or more genes that are differentially regulated in the sample as compared to a control sample is/are detected or identified, wherein the control sample comprises saliva RNA obtained from a neonate at a developmental stage different than the neonate from which the test sample of saliva RNA sample was obtained; and
determining that the one or more differentially regulated genes are involved in neonatal development.

5. The method of claim 4, wherein the developmental stage is assessed with respect to the neonate's feeding capability.

6. The method of claim 4, wherein the feeding capability is selected from the group consisting of readiness to feed, feeding tolerance, and combinations thereof.

7. The method of claim 1 or 4, wherein the test sample of saliva RNA comprises a plurality of nucleic acid segments labeled with a detectable agent and wherein the step of identifying comprises:
providing a gene-expression array comprising a plurality of genetic probes, wherein each genetic probe is immobilized to a discrete spot on a substrate surface to form an array;
contacting the array with the test sample under conditions wherein the nucleic acid segments in the sample specifically hybridize to the genetic probes on the array;
determining the binding of individual nucleic acid segments of the test sample to individual genetic probes immobilized on the array to obtain a binding pattern; and
establishing, based on the binding pattern obtained, a gene expression pattern.

8. A method for determining a diagnosis of a neonate comprising steps of:
providing a sample of saliva RNA obtained from the neonate;
subjecting the test sample of saliva RNA to an analysis, wherein the analysis comprises hybridizing the RNA to one or more oligonucleotide probes, such that expression of at least one gene identified using the method of claim 1 or 4 is identified;
and determining, based on the detected expression of the at least one gene, a diagnosis of the neonate.

9. The method of claim 8, wherein the step of determining a diagnosis comprises determining neonatal developmental progress.

10. The method of claim 9, wherein determining neonatal developmental progress comprises making a determination with respect to a feeding capability of the neonate.

11. The method of claim 10, wherein the feeding capability is selected from the group consisting of readiness to feed, feeding tolerance, and combinations thereof.

12. The method of claim 11, wherein the step of determining a diagnosis comprises identifying a disease or condition affecting the neonate.

13. The method of claim 12, wherein the disease or condition is necrotizing enterocolitis.

14. The method of claim 8, wherein the at least one gene is upregulated during neonatal development.

15. The method of claim 14, wherein the at least one gene is selected from the group consisting of neuropeptide Y receptor Y1 (NPY1R); leptin receptor (LEPR); growth hormone secretagogue receptor (GHSR); prostaglandin E receptor 3 (subtype EP3) (PTGER 3); hypocretin (orexin) receptor 2 (HCRTR2); galanin receptor 3 (GALR3); lactalbumin alpha (LALBA); glucagon (GCG); melanin-concentrating hormone receptor 1 (MCHR1); prostaglandin E receptor 3 (PTGER3); cholecytokinin A receptor (CCKAR); odorant binding protein 2B (OBP2B); transient receptor potential cation channel, subfamily V, member 1 (TRPV1); taste receptor, type 2, member 1 (TAS2R1); surfactant protein B (SFTPB); cystic fibrosis transmembrane conductance regulator (CFTR); fibroblast growth factors (FGF) 1, 2, 7, 10, 18; fibroblast growth receptor 2 (FGFR2); and combinations thereof.

16. The method of claim 8, wherein the at least one gene is downregulated during neonatal development.

17. The method of claim 16, wherein the at least one gene is selected from the group consisting of carcinoembryonic antigen-related cell adhesion molecule 1 (biliary glycoprotein) (CEACAM1); V-raf murine sarcoma viral oncogene homolog B1 (BRAF); amino-terminal enhancer of split (AES); E1A binding protein p300 (EP300); Fas (TNF receptor superfamily member 6) (FAS); Fas (TNFRSF6)-associated via death domain (FADD); cyclin-dependent kinase inhibitor 2A (melanoma, p16, inhibits CDK4) (CDKN2A); glycogen synthase kinase 3 Beta (GSK3B); protein kinase, cAMP-dependent, regulatory, type 1, alpha (tissue specific extinguisher 1) (PRKAR1A); signal transducer and activator of transcription 5B (STAT5B); aryl hydrocarbon receptor nuclear translocator (ARNT); insulin receptor (INSR); and combinations thereof.

18. The method of claim 8, further comprising detecting at least one gene selected from the group consisting of nuclear factor kappa B (NFκB), I kappa B-alpha (IκB-α), toll-like receptor 4 (TLR4), platelet activating factor (PAF), platelet activating factor acetylhydrolase (PAF-AH), interleukin 8 (IL-8), epidermal growth factor (EGF), interleukin 10 (IL-10), endothelial 1 (ET-1), and combinations thereof.
